(19) United States
(12) Patent Application Publication
(10) Pub. No.: US 2023/0000365 A1
(43) Pub. Date: Jan. 5, 2023

(54) FLUID CIRCUIT AND BLOOD PRESSURE MEASUREMENT DEVICE

(71) Applicant: OMRON HEALTHCARE Co., Ltd., Kyoto (JP)

(72) Inventors: Yuki TAKANO, Kyoto (JP); Yoshihide TOKKO, Kyoto (JP); Masaki HARADA, Kyoto (JP); Takashi ONO, Kyoto (JP)

(21) Appl. No.: 17/930,946

(22) Filed: Sep. 9, 2022

Related U.S. Application Data
(63) Continuation of application No. PCT/JP2021/010339, filed on Mar. 15, 2021.

(30) Foreign Application Priority Data
Mar. 16, 2020 (JP) 2020-045335

(51) Int. Cl. A61B 5/0225; A61B 5/0235; A61B 5/00; A61B 5/022

(52) U.S. Cl. CPC A61B 5/0225; A61B 5/0235; A61B 5/681; A61B 5/02233; A61B 2562/0247

(57) ABSTRACT

A fluid circuit of a blood pressure measurement device includes a first cuff connected to a secondary side of a pump that supplies a fluid to the secondary side, a first fluid resistor connected to a secondary side of the first cuff, a second fluid resistor provided on a secondary side of the first fluid resistor and connected to an atmosphere, and a second cuff provided between the first fluid resistor and the second fluid resistor.

### CROSS-REFERENCE TO RELATED APPLICATIONS

This application is the U.S. national stage application filed pursuant to 35 U.S.C. 365(c) and 120 as a continuation of International Patent Application No. PCT/JP2021/010339, filed Mar. 15, 2021, which application claims priority to Japanese Patent Application No. 2020-045335, filed Mar. 16, 2020, which applications are incorporated herein by reference in their entireties.

### TECHNICAL FIELD

The present invention relates to a fluid circuit and a blood pressure measurement device used for blood pressure measurement.

### BACKGROUND ART

In recent years, blood pressure measurement devices used for measuring a blood pressure are being used as means to check health status at home, as well as in medical facilities. A blood pressure measurement device detects vibration of the artery wall to measure blood pressure by, for example, inflating and contracting a cuff wrapped around the upper arm or the wrist of a living body and detecting the pressure of the cuff using a pressure sensor.

As such a blood pressure measurement device, there is known a technique that the blood pressure measurement device includes a sensing cuff for measuring a blood pressure and a plurality of cuffs including a pressing cuff that presses the sensing cuff against a living body. The blood pressure measurement device includes a pump and supplies a fluid, for example, air to the cuff by the pump to inflate the cuff

For example, JP 2009-22477 A discloses a technique of the blood pressure measurement device that disposes an orifice as a fluid resistor between a pressing cuff and a sensing cuff and includes a fluid circuit that reduces an amount of air injection. In such a blood pressure measurement device, a flow rate changes in proportion to a pressure difference between the pressing cuff on a primary side of the orifice and the sensing cuff on a secondary side of the orifice. Citation List—Patent Literature: Japanese Patent Application No.: JP 2009-22477 A.

### SUMMARY OF INVENTION

### Technical Problem

In the blood pressure measurement device described above, the flow rate of air supplied from a pump changes in proportion to the pressure difference between the pressing cuff on the primary side of the orifice and the sensing cuff on the secondary side of the orifice. Therefore, a change in pressurizing time of a living body during blood pressure measurement changes an amount of air inflow to the sensing cuff, causing an error in the amount of air injection to the sensing cuff

Similarly, in a configuration including three or more cuffs, providing a fluid resistor, such as an orifice, between the cuff on the primary side and the cuff on the secondary side changes the amount of air inflow to the cuff on the secondary side.

In addition, the pressurization time of the living body during blood pressure measurement changes depending on, for example, a thickness of a measurement site of a subject, a state of winding of an arm band, and a pump property. Further, the amount of air injection supplied to the sensing cuff needs to be smaller than an amount of intake air supplied to the pressing cuff. Therefore, as the orifice provided between the pressing cuff and the sensing cuff, an orifice having a large fluid resistance needs to be used. Such an orifice requires a fine pinhole and requires expensive and highly accurate processing technique.

Thus, an object of the present invention is to provide a fluid circuit and a blood pressure measurement device that allow controlling a pressure ratio of a plurality of cuffs.

### Solution To Problem

According to an aspect, there is provided a fluid circuit that includes a first cuff, a first fluid resistor, a second fluid resistor, and a second cuff. The first cuff is connected to a secondary side of a pump that supplies a fluid to a secondary side. The first fluid resistor is connected to a secondary side of the first cuff. The second fluid resistor is provided on a secondary side of the first fluid resistor and connected to an atmosphere. The second cuff is provided between the first fluid resistor and the second fluid resistor.

Here, the fluid includes a liquid and air. The cuff includes a bag-like structure that is wound around, for example, an upper arm and a wrist of a living body when a blood pressure is measured and inflates by supply of a fluid. When the fluid is air, the bag-like structure is an air bag inflated by, for example, air.

According to this aspect, the fluid supplied to the secondary side by the pump is supplied to the first cuff, passes through the first fluid resistor, and is supplied to a flow path between the first fluid resistor and the second fluid resistor. Further, the fluid supplied to the flow path between the first fluid resistor and the second fluid resistor is supplied to the second cuff, passes through the second fluid resistor, and is exhausted to the atmosphere. Thus, a flow rate of the fluid supplied to the second cuff becomes smaller than a flow rate of the fluid supplied to the first cuff. Furthermore, a flow rate of the fluid exhausted to the atmosphere becomes smaller than the flow rate of the fluid supplied to the second cuff. That is, a pressure ratio between a pressure of the first cuff and a pressure of the second cuff becomes constant by the use of a fluid resistance ratio between the first fluid resistor and the second fluid resistor. Thus, the fluid circuit allows setting the pressure of the first cuff and the pressure of the second cuff as desired pressures.

In the fluid circuit according to the one aspect described above, there is provided a fluid circuit that includes a first valve. The first valve is provided in parallel with the first fluid resistor. The first valve opens when a pressure of the second cuff is higher than a pressure of the first cuff by a predetermined value or more.

According to this aspect, in the fluid circuit, in a case where the pressure of the first cuff decreases due to, for example, exhaust of the fluids of the first cuff and the second cuff, when the pressure of the first cuff becomes lower than the pressure of the second cuff, the first valve opens. Thus, when the pressure of first cuff is higher than the pressure of the second cuff, the fluid of the first cuff is preferentially exhausted, and the fluid of the second cuff is exhausted through the respective first fluid resistor and second fluid resistor. Additionally, when the pressure of the first cuff becomes lower than the pressure of the second cuff, the first valve opens and an exhaust rate of the fluid of the second cuff increases.

In the fluid circuit according to the aspect described above, there is provided a fluid circuit that includes a second valve and a third fluid resistor. The second valve is provided in parallel with the first fluid resistor. The second valve opens when a pressure of the first cuff is higher than a pressure of the second cuff by a predetermined value or more. The third fluid resistor is connected to a secondary side of the second valve. The third fluid resistor is provided in parallel with the first fluid resistor.

According to this aspect, as the pressure of the first cuff increases, a differential pressure with the pressure of the second cuff increases. However, the differential pressure between the first cuff and the second cuff can be reduced by the use of the second valve and the third fluid resistor provided in parallel with the first fluid resistor.

Thus, the fluid circuit can increase the second cuff to a desired pressure without requiring a pump having a high capacity. In the fluid circuit according to the aspect described above, there is provided a fluid circuit in which at least one of the first fluid resistor and the second fluid resistor is formed by connecting a plurality of orifices in parallel, in series, or in series and parallel. According to this aspect, by setting an arrangement of a plurality of flow rate resistors, the fluid resistors used for fluid resistance can be the fluid resistors taking into account pressure dependence.

According to an aspect, there is provided a blood pressure measurement device that includes a pump, a fluid circuit, an on-off valve, a pressure sensor, and a control unit. The pump supplies a fluid to a secondary side. The fluid circuit is according to any one of the aspects described above. The on-off valve is provided between the pump and the first cuff. The on-off valve opens and closes a flow path to the atmosphere. The pressure sensor is connected to the second cuff. The control unit that controls the pump and the on-off valve based on a pressure detected by the pressure sensor.

According to this aspect, the blood pressure measurement device can drive the pump based on the pressure of the second cuff, and thus the fluid can be supplied to the second cuff until at least the second cuff reaches a preferred pressure. Further, when the blood pressure measurement device exhausts the fluids of the first cuff and the second cuff, the fluids can be exhausted from the first cuff and the second cuff by controlling and opening the on-off valve by the control unit.

In the blood pressure measurement device according to the aspect described above, there can be provided a blood pressure measurement device that includes a device body. The device body houses the pump, the on-off valve, the pressure sensor, and the control unit. The first fluid resistor and the second fluid resistor are integrally provided with the first cuff.

According to this aspect, in the blood pressure measurement device, the device body houses the control unit, the pump controlled by the control unit, the on-off valve, and the pressure sensor. The first valve and the fluid resistor that are used for fluid control of the fluid circuit and not electrically connected to the control unit are integrally provided with the first cuff, and the device body does not house the first valve or the fluid resistor. Thus, the device body of the blood pressure measurement device can be miniaturized.

### Advantageous Effects Of Invention

The present invention can provide the fluid circuit and the blood pressure measurement device that allow controlling the pressure ratio of the plurality of cuffs.

### BRIEF DESCRIPTION OF DRAWINGS

FIG. 1 is an explanatory view schematically illustrating a configuration of a blood pressure measurement device according to a first embodiment of the present invention;

FIG. 2 is a block diagram schematically illustrating a configuration of a device body of the blood pressure measurement device;

FIG. 3 is a block diagram illustrating the configuration of the blood pressure measurement device and illustrating an example of usage of the blood pressure measurement device;

FIG. 4 is a block diagram illustrating the configuration of the blood pressure measurement device and illustrating an example of usage of the blood pressure measurement device;

FIG. 5 is an explanatory view illustrating an example of a change in pressure and a change in amount of injection in blood pressure measurement by the blood pressure measurement device;

FIG. 6 is a flowchart depicting an example of usage of the blood pressure measurement device;

FIG. 7 is an explanatory view schematically illustrating a configuration of a blood pressure measurement device according to a second embodiment of the present invention;

FIG. 8 is a block diagram illustrating the configuration of the blood pressure measurement device and illustrating an example of usage of the blood pressure measurement device;

FIG. 9 is a block diagram illustrating the configuration of the blood pressure measurement device and illustrating an example of usage of the blood pressure measurement device;

FIG. 10 is an explanatory view schematically illustrating a configuration of a blood pressure measurement device according to a third embodiment of the present invention;

FIG. 11 is a block diagram illustrating the configuration of the blood pressure measurement device and illustrating an example of usage of the blood pressure measurement device;

FIG. 12 is a block diagram illustrating the configuration of the blood pressure measurement device and illustrating an example of usage of the blood pressure measurement device;

FIG. 13 is a perspective view illustrating a configuration of a blood pressure measurement device according to a fourth embodiment of the present invention;

FIG. 14 is a plan view illustrating a configuration of a cuff structure and a fluid control unit of the blood pressure measurement device;

FIG. 15 is an explanatory view schematically illustrating a configuration of a blood pressure measurement device according to a fifth embodiment of the present invention;

FIG. 16 is an explanatory view schematically illustrating a configuration of a blood pressure measurement device according to a sixth embodiment of the present invention;

FIG. 17 is an explanatory view schematically illustrating a configuration of a blood pressure measurement device according to an eighth embodiment of the present invention;

FIG. 18 is an explanatory view schematically illustrating a configuration of a blood pressure measurement device according to a ninth embodiment of the present invention;

FIG. 19 is an explanatory view schematically illustrating a configuration of a blood pressure measurement device according to a tenth embodiment of the present invention;

FIG. 20 is an explanatory view schematically illustrating a configuration of a blood pressure measurement device according to an eleventh embodiment of the present invention;

FIG. 21 is an explanatory view schematically illustrating a configuration of a blood pressure measurement device according to a twelfth embodiment of the present invention;

FIG. 22 is an explanatory view schematically illustrating a configuration of a modified example of the blood pressure measurement device according to the twelfth embodiment of the present invention;

FIG. 23 is a cross-sectional view illustrating a configuration of a fluid resistor used in the blood pressure measurement device and illustrating an example in which a plurality of flow rate resistors are disposed in parallel;

FIG. 24 is a plan view illustrating a configuration of an orifice film used for the fluid resistor;

FIG. 25 is a cross-sectional view illustrating a configuration of another example of a fluid resistor and illustrating an example in which a plurality of flow rate resistors are disposed in series;

FIG. 26 is a plan view illustrating a configuration of the orifice film used for the fluid resistor;

FIG. 27 is a cross-sectional view illustrating a configuration of another example of the fluid resistor;

FIG. 28 is a cross-sectional view illustrating an enlarged configuration of the fluid resistor; and,

FIG. 29 is an explanatory view schematically illustrating a configuration of another modified example of the blood pressure measurement device according to the twelfth embodiment of the present invention.

### DESCRIPTION OF EMBODIMENTS

### First Embodiment

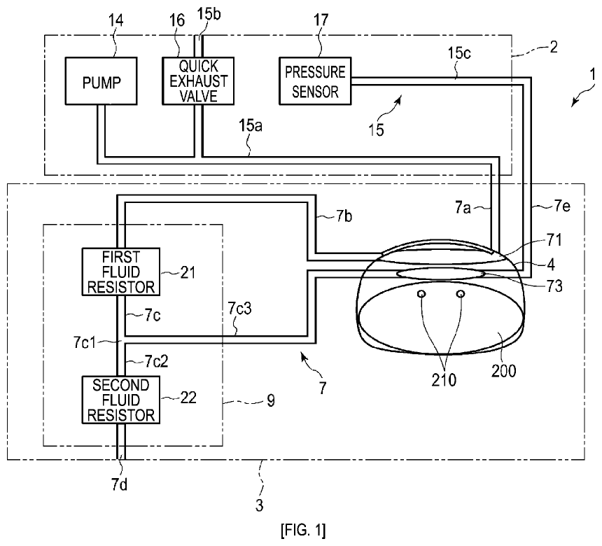

An example of a blood pressure measurement device 1 according to the first embodiment of the present invention will be described below with reference to FIG. 1 to FIG. 6.

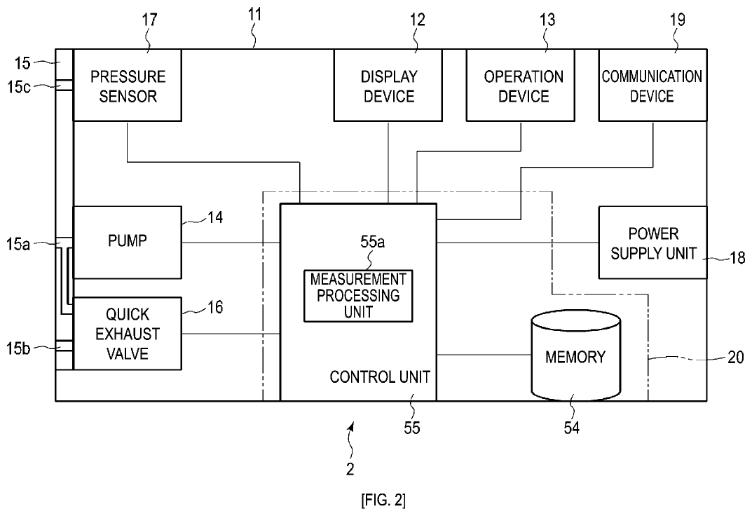

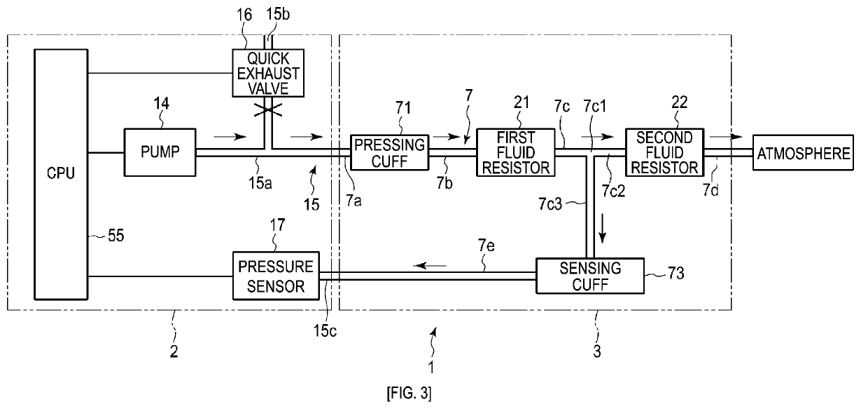

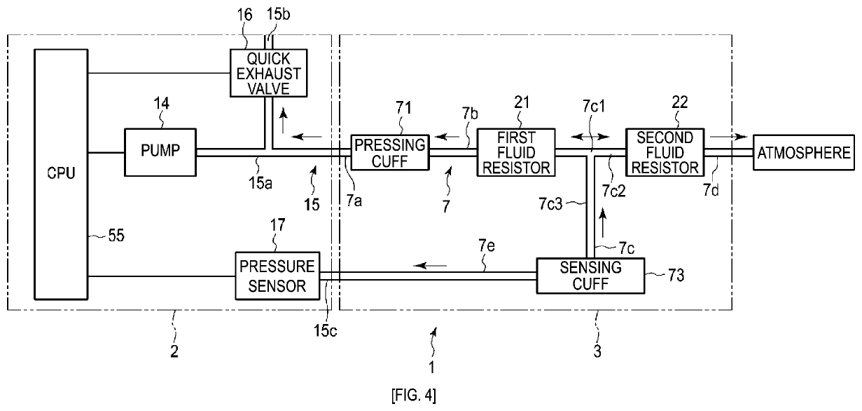

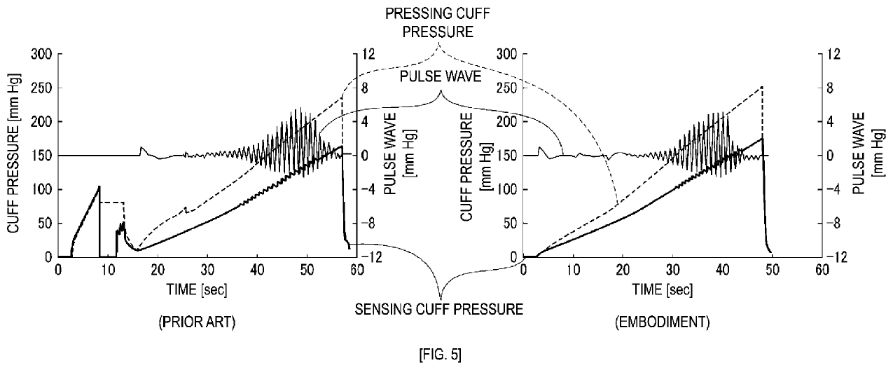

FIG. 1 is an explanatory view schematically illustrating a configuration of the blood pressure measurement device 1 according to the first embodiment of the present invention. FIG. 2 is a block diagram schematically illustrating a configuration of a device body 2 of the blood pressure measurement device 1. FIG. 3 is a block diagram illustrating the configuration of the blood pressure measurement device 1 and illustrating an example of a flow of a fluid supplied to each of cuffs 71 and 73 in blood pressure measurement. FIG. 4 is a block diagram illustrating the configuration of the blood pressure measurement device 1 and illustrating an example of a flow of a fluid in exhaust of the fluid after blood pressure measurement. FIG. 5 is an explanatory view illustrating an example of a change in pressure and a change in amount of injection of each of the cuffs 71 and 73 in blood pressure measurement by the blood pressure measurement device 1.

The blood pressure measurement device 1 is an electronic blood pressure measurement device attached to a living body. The blood pressure measurement device 1 is an electronic blood pressure measurement device that is attached to a living body 200, such as a wrist, and has an aspect of measuring a blood pressure from arteries 210 of the living body 200. As illustrated in FIG. 1, FIG. 3, and FIG. 4, the blood pressure measurement device 1 includes the device body 2 and a fluid circuit 3. For example, as illustrated in FIG. 1, the blood pressure measurement device 1 includes a fixture 4, such as a belt, that fixes at least the fluid circuit 3 to the living body 200. Note that FIG. 1 illustrates a wrist as the living body 200, but the living body 200 may be, for example, an upper arm.

As illustrated in FIG. 2, the device body 2 includes a case 11, a display device 12, an operation device 13, a pump 14, a flow path unit 15, an on-off valve 16, a pressure sensor 17, a power supply unit 18, a communication device 19, and a control substrate 20.

The case 11 houses, for example, the display device 12, the operation device 13, the pump 14, the flow path unit 15, the on-off valve 16, the pressure sensor 17, the power supply unit 18, the communication device 19, and the control substrate 20. Additionally, the case 11 exposes a portion of the display device 12 or is made of a transparent material such that a portion of the display device 12 can be visually recognized from the outside. Note that the case 11 may be configured to house a portion of the configuration of the fluid circuit 3.

The display device 12 is electrically connected to the control substrate 20. The display device 12 is, for example, a Liquid Crystal Display (LCD) or an Organic Electro Luminescence Display (OELD). The display device 12 displays various types of information including date and time and measurement results of, for example, blood pressure values, such as a systolic blood pressure and a diastolic blood pressure, and a heart rate in response to a control signal from the control substrate 20.

A user inputs an instruction with the operation device 13. For example, the operation device 13 is a sensor that includes a plurality of buttons and detects an operation of the button, for example, a pressure-sensitive or capacitive touch panel provided on, for example, the case 11 and the display device 12, and a microphone for receiving an instruction by sound. When operated by the user, the operation device 13 converts the instruction into an electrical signal and outputs the electrical signal to the control substrate 20.

The pump 14 is, for example, a piezoelectric pump. The pump 14 compresses a fluid and supplies the compressed fluid to the fluid circuit 3 through the flow path unit 15. The pump 14 is electrically connected to the control substrate 20. The pump 14 drives based on the control signal provided from the control substrate 20. Here, any gas or any liquid can be employed as the fluid. In the present embodiment, the fluid is air.

The flow path unit 15 connects the pump 14, the on-off valve 16, and the pressure sensor 17 to the fluid circuit 3. The flow path unit 15 is any of, for example, a tube, a pipe, a tank, and a hollow portion and a groove formed in the case 11, or a combination thereof. As a specific example, the flow path unit 15 forms a flow path from the pump 14 to a secondary side, and forms a flow path 15a that is formed by branching a portion of the flow path from the pump 14 to the secondary side. Thus, the flow path 15a connects to the on-off valve 16. The flow path unit 15 forms a flow path 15b that connects the on-off valve 16 to an atmosphere. The flow path unit 15 forms a flow path 15c that connects the pressure sensor 17 to the fluid circuit 3.

The on-off valve 16 is electrically connected to the control substrate 20. The on-off valve 16 is controlled by the control substrate 20. For example, the on-off valve 16 is opened and closed by control of the control substrate 20. The on-off valve 16 is connected to the atmosphere by the flow path unit 15 and is switched to an open state to connect the pump 14 and the fluid circuit 3 to the atmosphere.

The on-off valve 16 is an exhaust valve that opens the flow path on the secondary side of the pump 14 to the atmosphere. In addition, for example, the on-off valve 16 is, for example, a quick exhaust valve to set an opening degree of the on-off valve 16 or an opening area of the flow path unit 15 such that a fluid resistance becomes low as much as possible and allows quick exhaust. Note that each of the drawings illustrates the on-off valve 16 as the quick exhaust valve 16. The on-off valve 16 is switched to a closed state when air is supplied to the fluid circuit 3 during blood pressure measurement. In addition, when the fluid circuit 3 is exhausted, the on-off valve 16 is controlled by the control substrate 20 so as to be switched from the closed state to the open state. Further, the on-off valve 16 may be formed such that the opening degree is adjustable.

The pressure sensor 17 detects a pressure of the cuff disposed on the secondary side of the fluid circuit 3, and in the present embodiment, a pressure of a sensing cuff 73 described later of the fluid circuit 3. As a specific example, the pressure sensor 17 is fluidly connected to the sensing cuff 73 via the flow path unit 15, and detects the pressure inside the sensing cuff 73. The pressure sensor 17 is electrically connected to the control substrate 20. The pressure sensor 17 outputs an electrical signal corresponding to the detected pressure to the control substrate 20.

The power supply unit 18 is a power source. The power supply unit 18 is, for example, a secondary battery, such as a lithium ion battery. The power supply unit 18 is electrically connected to the control substrate 20. As a specific example, the power supply unit 18 supplies power to the control substrate 20. The power supply unit 18 supplies driving power to the respective configurations of the control substrate 20, the display device 12, the operation device 13, the pump 14, the on-off valve 16, the pressure sensor 17, and the communication device 19 via the control substrate 20.

The communication device 19 can transmit and receive information to and from an external device wirelessly or by wire. The communication device 19 transmits information, such as information controlled by the control substrate 20 and measured blood pressure values and pulse, to an external device, or receives, for example, a program for software update from an external device and transmits this to the control unit.

In the present embodiment, the external device is, for example, an external terminal, such as a smartphone, a tablet terminal, a personal computer, and a smart watch.

In the present embodiment, the communication device 19 and the external device may be directly connected, or may be connected over a network. The communication device 19 and the external device may be connected via a mobile communication network, such as 4G and 5G, and a wireless communication line, such as Wimax and Wi-Fi (registered trademark). Further, the communication device 19 and the external device may be connected by wireless communication means, such as Bluetooth (registered trademark), Near Field Communication (NFC), and infrared communication. Furthermore, the communication device 19 and the external device may be connected over a wired communication line, such as a Universal Serial Bus (USB) and a Local Area Network (LAN) connection with a cable. Thus, the communication device 19 may include a plurality of communication means, such as a wireless antenna and a micro-USB connector.

The control substrate 20 includes, for example, a substrate, a storage unit 54, and a control unit 55. The control substrate 20 is constituted by mounting the storage unit 54 and the control unit 55 on a substrate.

The substrate is fixed to the case 11.

The storage unit 54 is a memory mounted on the substrate. The storage unit 54 includes, for example, a Random Access Memory (RAM) and a Read Only Memory (ROM). The storage unit 54 stores various types of data. For example, the storage unit 54 pre-stores, for example, program data for controlling the overall blood pressure measurement device 1, the pump 14, and the fluid circuit 3, settings data for setting various functions of the blood pressure measurement device 1, and calculation data for calculating a blood pressure value and a pulse from the pressure measured by the pressure sensor 17 to be changeable. The storage unit 54 stores information, such as a measured blood pressure value, a measured value of, for example, a pulse, and a pressure value measured by the pressure sensor 17. The storage unit 54 can store various types of data generated by a measurement processing unit 55a in the control unit 55.

The control unit 55 includes a single or a plurality of processors mounted on the substrate. For example, the processor is a Central Processing Unit (CPU). The control unit 55 controls the operation of the entire blood pressure measurement device 1 and the operations of the pump 14 and the fluid circuit 3 based on the programs stored in the storage unit 54 to perform a predetermined operation (function). In addition, in accordance with the read program, the control unit 55 performs, for example, predetermined operation, analysis, or process in the control unit 55.

The control unit 55 is electrically connected to and supplies power to the display device 12, the operation device 13, the pump 14, the on-off valve 16, and the pressure sensor 17. Additionally, the control unit 55 controls the operations of the display device 12, the pump 14, and the on-off valve 16, based on electrical signals output by the operation device 13 and the pressure sensor 17.

For example, the control unit 55 includes a main CPU that controls the operation of the overall blood pressure measurement device 1 and a sub-CPU that controls the operation of the fluid circuit 3. Note that, for example, the control unit 55 may be configured to perform all the controls of the blood pressure measurement device 1 in one CPU. For example, the main CPU obtains measurement results, such as blood pressure values, for example, the systolic blood pressure and the diastolic blood pressure, and the heart rate, from the electrical signals output by the pressure sensor 17, and outputs image signals corresponding to the measurement results to the display device 12.

For example, the sub-CPU drives the pump 14 and the on-off valve 16 to feed compressed air to the fluid circuit 3 when an instruction to measure the blood pressure is input from the operation device 13. In addition, the sub-CPU controls driving and stop of the pump 14 and opening and closing of the on-off valve 16 based on the electrical signal output by the pressure sensor 17. The sub-CPU controls the pump 14 and the on-off valve 16 to supply the compressed air to the fluid circuit 3 and selectively depressurize the fluid circuit 3.

Thus, the control unit 55 configures a portion of all of the respective functions performed by the control unit 55 in hardware by, for example, one or a plurality of integrated circuits. For example, the control unit 55 includes the measurement processing unit 55a. For example, the measurement processing unit 55a controls the pump 14 and the on-off valve 16 to supply air to the fluid circuit 3, and calculates the blood pressure by an oscillometric method based on the pressure of the sensing cuff 73 described later of the fluid circuit 3 detected by the pressure sensor 17.

The fluid circuit 3 includes a cuff structure 6, a tube group 7, and a fluid control unit 9. The fluid circuit 3 fluidly connects the cuff structure 6 and the fluid control unit 9 by the tube group 7.

Note that when air is supplied from the pump 14 to the fluid circuit 3, in the air flow, the pump 14 side (the device body 2 side) becomes the primary side and the fluid circuit 3 side becomes the secondary side. However, during exhaust, the on-off valve 16 side (the device body 2 side) becomes the secondary side and the fluid circuit 3 side becomes the primary side. However, in the description of the configuration of the fluid circuit 3, for convenience of explanation, the primary side and the secondary side are defined based on the flow direction of air when the air is supplied from the pump 14 to the cuff structure 6 and the tube group 7.

The cuff structure 6 includes a plurality of cuffs. Here, the cuff is wrapped around, for example, a wrist of a living body to measure a blood pressure and includes a single or multi-layer bag-like structures that are inflated by being supplied with a fluid. The bag-like structure is inflated by fluid. In the present embodiment, since the fluid is air, the bag-like structure is an air bag. The bag-like structure is formed, for example, by stacking and welding a pair of sheet members.

For example, the cuff structure 6 includes a first cuff 71 and the second cuff 73. The first cuff 71 is fluidly connected to the pump 14. The first cuff 71 is inflated by the air from the pump 14. The first cuff 71 is a pressing cuff that is inflated to press the second cuff 73 against the living body. Hereinafter, the first cuff 71 will be described as the pressing cuff 71. The pressing cuff 71 is formed by, for example, stacking a plurality of fluidly connected air bags in the pressing direction of the second cuff 73.

The second cuff 73 is provided on the secondary side of the first cuff 71. The second cuff 73 is inflated by air from the pump 14. The second cuff 73 is disposed in a region where the arteries 210 are present in the living body 200 when the blood pressure measurement device 1 is attached to the living body. The second cuff 73 is a sensing cuff for calculating the blood pressure in blood pressure measurement. Hereinafter, the second cuff 73 will be described as the sensing cuff 73. The sensing cuff 73 is supplied with air and is pressed by the inflated pressing cuff 71 to compress the region where the arteries 210 are present in the living body 200 through the use of the inflated pressing cuff 71. The sensing cuff 73 is pressed to the wrist 200 side by the inflated pressing cuff 71. The sensing cuff 73 is formed by, for example, one air bag. The sensing cuff 73 is fluidly connected to the pressing cuff 71 via the fluid control unit 9. In the present embodiment, an example in which the sensing cuff 73 is fluidly connected to the secondary side of the pressing cuff 71 via the fluid control unit 9 will be described.

The tube group 7 is a collection of, for example, tubes or hollow portions provided between the sheet members constituting the air bag. The tube group 7, for example, may be integrally provided with the cuff structure 6, or may be separated from the cuff structure 6 and connected to the cuff structure 6.

The tube group 7 fluidly connects the pressing cuff 71, the sensing cuff 73, and the fluid control unit 9. The tube group 7 is connected to the flow path unit 15. In the present embodiment, an example of the tube group 7 in a configuration in which the fluid control unit 9 includes a first fluid resistor 21 and a second fluid resistor 22 will be described.

The tube group 7, for example, fluidly connects the pump 14, the on-off valve 16, and the pressing cuff 71 via the flow path unit 15. The tube group 7, for example, fluidly connects the pressure sensor 17 and the sensing cuff 73 via the flow path unit 15. Also, for example, the tube group 7 fluidly connects the first fluid resistor 21, the second fluid resistor 22, and the atmosphere in series on the secondary side of the pressing cuff 71 and fluidly connects the sensing cuff 73 between the first fluid resistor 21 and the second fluid resistor 22.

Specifically, the tube group 7 includes a first tube 7a, a second tube 7b, a third tube 7c, a fourth tube 7d, and a fifth tube 7e. The first tube 7a is connected to the flow path 15a of the flow path unit 15 and the pressing cuff 71. The first tube 7a connects the pump 14, the on-off valve 16, and the pressing cuff 71 via the flow path unit 15.

The second tube 7b is connected to the pressing cuff 71 and the first fluid resistor 21. The third tube 7c has a branch portion 7c1 in the middle portion from the primary side toward the secondary side, and is a branch pipe branched into two flow paths at the branch portion 7c1. The primary side of the third tube 7c is fluidly connected to the first fluid resistor 21. One branched tube portion 7c2 on the secondary side of the third tube 7c is connected to the second fluid resistor 22. Another branched tube portion 7c3 on the secondary side of the third tube 7c and the is connected to the sensing cuff 73.

The fourth tube 7d connects the second fluid resistor 22 and the atmosphere.

That is, the fourth tube 7d has a first end connected to the secondary side of the second fluid resistor 22 and a second end opening to the outside. The fifth tube 7e is connected to the flow path 15c of the flow path unit 15 and the sensing cuff 73. The fifth tube 7e connects the pressure sensor 17 and the sensing cuff 73 via the flow path unit 15.

The fluid control unit 9 controls a pressure ratio of the air between the two cuffs 71 and 73 so as to be constant by a fluid resistance ratio between the two fluid resistors 21 and 22. As illustrated in FIG. 1, FIG. 4, and FIG. 5, the fluid control unit 9 is provided between the pressing cuff 71 and the sensing cuff 73, for example.

The fluid control unit 9 includes the first fluid resistor 21 and the second fluid resistor 22 connected in series. The fluid control unit 9 generates pressure differences between the pressure on the primary side of the first fluid resistor 21, the pressure between the first fluid resistor 21 and the second fluid resistor 22, and the pressure on the secondary side of the second fluid resistor 22. The fluid control unit 9 controls the pressure ratio between the pressing cuff 71 on the primary side of the first fluid resistor 21 and the sensing cuff 73 connected between the first fluid resistor 21 and the second fluid resistor 22 so as to be constant by these pressure differences.

The first fluid resistor 21 provides a resistance of the passing fluid, air in the present embodiment. The first fluid resistor 21 has, for example, a flow path cross-sectional area smaller than flow path cross-sectional areas on the primary side and the secondary side of the first fluid resistor 21, that is, flow path cross-sectional areas of the tube portion 7c2 of the third tube 7c and the fourth tube 7d. The first fluid resistor 21 is, for example, orifices. The first fluid resistor 21 reduces the flow path on the flow path from the pressing cuff 71 to the sensing cuff 73 to lower a flow rate of air supplied to the secondary side of the first fluid resistor 21 than a flow rate of air supplied to the pressing cuff 71.

The second fluid resistor 22 provides a resistance of the passing fluid, air in the present embodiment. The second fluid resistor 22 has, for example, a flow path cross-sectional area smaller than flow path cross-sectional areas on the primary side and the secondary side of the second fluid resistor 22, that is, flow path cross-sectional areas of the second tube 7b and the third tube 7c. The second fluid resistor 22 is, for example, orifices.

The second fluid resistor 22 reduces the flow path from the flow path between the first fluid resistor 21 and the second fluid resistor 22 to the flow path to the atmosphere to lower a flow rate of air supplied to the secondary side (the atmosphere) of the second fluid resistor 22 than a flow rate of air supplied to the first fluid resistor 21 and the second fluid resistor 22. That is, when a portion of the air supplied to the flow path between the first fluid resistor 21 and the second fluid resistor 22 flows to the sensing cuff 73 and the atmosphere, the second fluid resistor 22 provides a resistance of air flow toward the atmosphere side, and controls the flow rate of air injected into the sensing cuff 73 and the flow rate of air flowing out to the atmosphere.

For a fluid resistance ratio between the first fluid resistor 21 and the second fluid resistor 22, for example, by experimentally obtaining a relationship between the resistance ratio between the first fluid resistor 21 and the second fluid resistor 22 and a measurement error, the optimal fluid resistance ratio is set. Note that the optimal fluid resistance ratio is, for example, a fluid resistance ratio that ideally becomes “compression force loss=differential pressure=pressing cuff pressure−sensing cuff pressure” by compression to the arteries 210 by the pressing cuff 71 in the blood pressure measurement device 1 wound around the wrist 200.

A specific example of the setting of the fluid resistance ratio is that the blood pressure is measured multiple times with the fluid resistance ratios between the first fluid resistor 21 and the second fluid resistor 22 differentiated, the respective measurement errors are obtained, and the optimal fluid resistance ratio is estimated from the blood pressure measurement errors. For example, assume that the blood pressure error is approximately 5 mm Hg with a first fluid resistance ratio (the first fluid resistor 21/second fluid resistor 22) of 0.67 and the blood pressure error is approximately −15 mm Hg with a second fluid resistance ratio of 1. From the relationship, the optimal fluid resistance ratio where the blood pressure error becomes 0 mm Hg can be estimated as 0.75. Then, the first fluid resistor 21 and the second fluid resistor 22 to obtain the fluid resistance ratio are set. Note that the relationship of the fluid resistance ratio between first fluid resistor 21 and the second fluid resistor 22 changes depending on the compression force by the cuffs 71 and 73 of the blood pressure measurement device 1, and therefore adjustment is performed according to the properties of the cuffs 71 and 73.

Next, an example of the change in pressures of the pressing cuff 71 and the sensing cuff 73 when air is supplied to the fluid circuit 3 will be described with reference to FIG. 3 and FIG. 5. FIG. 3 indicates the air flow by the arrows and a flow path where each valve closes by X.

In the fluid circuit 3, the on-off valve 16 is closed by the measurement processing unit 55a in the control unit 55 during blood pressure measurement, and when the pump 14 starts driving, the air is first supplied to the pressing cuff 71. A portion of the air supplied to the pressing cuff 71 passes through the first fluid resistor 21 and is supplied to the flow path between the first fluid resistor 21 and the second fluid resistor 22. At this time, a portion of the air supplied to the pressing cuff 71 according to the resistance of the first fluid resistor 21 is supplied to the flow path between the first fluid resistor 21 and the second fluid resistor 22.

A portion of the air supplied to the flow path between the first fluid resistor 21 and the second fluid resistor 22 is injected into the sensing cuff 73, and the other air supplied to the flow path between the first fluid resistor 21 and the second fluid resistor 22 passes through the second fluid resistor 22 and is exhausted to the atmosphere. At this time, due to the resistance of the second fluid resistor 22, the flow rate of the air flowing to each of the sensing cuff 73 and the atmosphere changes in the air supplied to the flow path between the first fluid resistor 21 and the second fluid resistor 22.

In addition, since the fluid resistance ratio of the first fluid resistor 21 and the second fluid resistor 22 is set, as illustrated in FIG. 5 (the embodiment), the pressure ratio between the pressure of the pressing cuff 71 and the pressure of the sensing cuff 73 is constant from the start of supply of the air by the pump 14 until the stop of supply of the air. Specifically, the one (prior art) illustrated in FIG. 5 is an example of the change in pressure of the blood pressure measurement device according to the prior art. In the blood pressure measurement device according to the prior art, each of cuffs is provided with an on-off valve, each of the on-off valves is opened and closed by the measurement processing unit 55a, and the pressure of each of the cuffs is controlled by a pressure sensor connected to each of the cuffs. Therefore, adjustment is required to control each of the on-off valves at the start of blood pressure measurement. On the other hand, the fluid control unit 9 of the present embodiment utilizes partial pressures of the first fluid resistor 21 and the second fluid resistor 22, and thus only the supplied air passing through the first fluid resistor 21 and the second fluid resistor 22 makes the pressure ratio from the start of supply of air until the stop of supply of air constant.

Next, an example of the change in pressures of the pressing cuff 71 and the sensing cuff 73 when the air supplied to the fluid circuit 3 is exhausted will be described with reference to FIG. 4. FIG. 4 indicates the air flow by the arrows.

In the fluid circuit 3, when the exhaust of the fluid circuit 3 starts after blood pressure measurement, the measurement processing unit 55a in the control unit 55 stops the pump 14 and opens the on-off valve 16. As a result, since the on-off valve 16 side of the pressing cuff 71 is connected to the atmosphere, the air in the pressing cuff 71 flows to the on-off valve 16 side. The air in the sensing cuff 73 is exhausted to the atmosphere via the second fluid resistor 22. When the exhaust of the pressing cuff 71 progresses, the pressure of the pressing cuff 71 decreases, and when the pressure of the pressing cuff 71 is lower than the pressure of the sensing cuff 73, in addition to the exhaust to the atmosphere via the second fluid resistor 22, the air in the sensing cuff 73 moves to the pressing cuff 71 through the first fluid resistor 21 and is exhausted from the on-off valve 16 to the atmosphere. Thus, the fluid circuit 3 is exhausted.

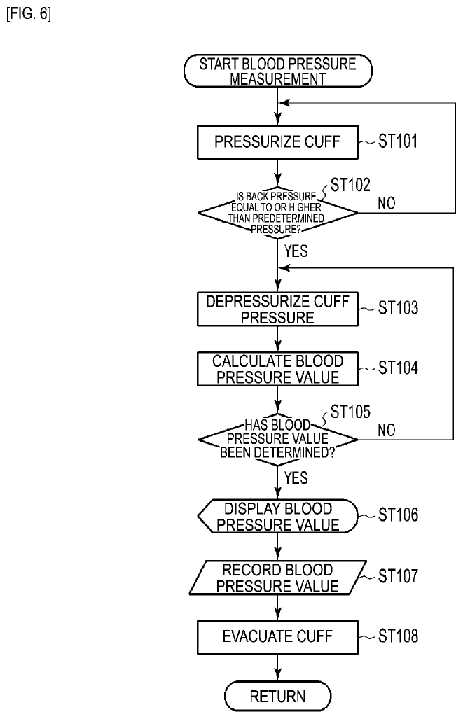

Next, an example of the control during blood pressure measurement using the blood pressure measurement device 1 configured in this manner will be described with reference to the flow chart depicted in FIG. 6.

First, with the blood pressure measurement device 1 attached to the living body 200, the user operates the operation device 13 to perform the instruction to start the blood pressure measurement. The operation device 13 outputs the electrical signal to the control unit 55 as the instruction to start the blood pressure measurement. When the control unit 55 receives the electrical signal from the operation device 13, the measurement processing unit 55a switches the on-off valve 16 to the closed state, starts driving the pump 14, and pressurizes the pressing cuff 71 and the sensing cuff 73 (step ST101). Then, the measurement processing unit 55a determines whether the pressure measured by the pressure sensor 17 is a predetermined pressure (step ST102). Here, the predetermined pressure is the pressure of the sensing cuff 73 at which the blood pressure can be measured by the sensing cuff 73 and is stored in the storage unit 54 in advance.

When the pressure of the sensing cuff 73 is not the predetermined pressure (NO in step ST102), the measurement processing unit 55a continues driving the pump 14. When the pressure of the sensing cuff 73 reaches the predetermined pressure (YES in step ST102), the measurement processing unit 55a stops the pump 14 and stops supplying air to the pressing cuff 71. Furthermore, the measurement processing unit 55a switches the on-off valve 16 to the open state and starts depressurizing the pressing cuff 71 (step ST103). At this time, the measurement processing unit 55a adjusts the degree of opening of the on-off valve 16 or repeatedly switches the opening/closing of the on-off valve 16, and thus the pressing cuff 71 is depressurized gently.

The measurement processing unit 55a calculates the blood pressure value from the pressure measured by the pressure sensor 17 (step ST104). Next, the measurement processing unit 55a determines whether the calculated value should be determined as the blood pressure value (step ST105). Note that a threshold value for whether the calculated value should be determined as the blood pressure value is stored in the storage unit 54 in advance. Also, the threshold value for determining the blood pressure value is appropriately set by, for example, the detected blood pressure value and the pressure of the sensing cuff 73. When the calculated value cannot be determined as the blood pressure value (NO in step ST105), the measurement processing unit 55a continues depressurizing the pressing cuff 71 (step ST103). In a case where the calculated value is determined as the blood pressure value (YES in step ST105), the measurement processing unit 55a displays the blood pressure value on the display device 12 (step ST106), and records (stores) the measured blood pressure value in the storage unit 54 (step ST107). The measurement processing unit 55a is then maximizes the degree of opening of the on-off valve 16 or sets the on-off valve 16 in the open state, and exhausts the exhaust cuff 71 and the sensing cuff 73 (step ST108). Then, the measurement processing unit 55a ends the blood pressure measurement and stands by for the next instruction to start blood pressure measurement. When the measurement processing unit 55a receives the instruction to start blood pressure measurement, returns the step again to step ST101, and starts the blood pressure measurement.

According to the blood pressure measurement device 1 configured in this manner, the fluid circuit 3 provides the first fluid resistor 21 and the second fluid resistor 22 on the secondary side of the pressing cuff 71, the sensing cuff 73 is connected to the flow path between the first fluid resistor 21 and the second fluid resistor 22, and the secondary side of the second fluid resistor 22 is connected to the atmosphere. With this configuration, when air is supplied by the pump 14 in the blood pressure measurement, the pressure ratio between the pressing cuff 71 and the sensing cuff 73 is constant. This eliminates the need for the step of injection process of air by the measurement processing unit 55a in the control unit 55 in the blood pressure measurement, thus shortening the measurement time. Therefore, an influence of artifact, such as body movement, is solved, and the blood pressure measurement device 1 improves in robustness in actual use.

The pressure difference between the pressing cuff 71 and the sensing cuff 73 can be set by the fluid resistance ratio between the first fluid resistor 21 and the second fluid resistor 22. As a result, by only the air passing through the first fluid resistor 21 and the second fluid resistor 22, the pressing cuff 71 and the sensing cuff 73 inflate at the preferred pressure ratio. Thus, the blood pressure measurement device 1 does not require a component that is electrically controlled by, for example, the control unit 55 other than the pump 14 or the on-off valve 16 to make the pressure ratio between the pressing cuff 71 and the sensing cuff 73 constant.

Thus, the blood pressure measurement device 1 can simplify the control in blood pressure measurement and reduce the power consumption. Furthermore, the first fluid resistor 21 or the second fluid resistor 22 that does not require electrical control need not be disposed in the device body 2, and providing the fluid control unit 9 outside the device body 2 allows miniaturizing the device body 2.

Furthermore, as illustrated in FIG. 5, the blood pressure measurement device 1 can have the constant pressure ratio from the start of driving the pump 14 in comparison to a case where on-off valves are provided on respective primary sides of the pressing cuff 71 and the sensing cuff 73 of the conventional blood pressure measurement device and each of the on-off valves is controlled based on the detected pressure. Thus, the time from the start of driving the pump 14 until the measurement of blood pressure from can be shortened.

Furthermore, during exhaust of the fluid circuit 3, the air in the sensing cuff 73 is exhausted by the two flow paths, exhausted to the atmosphere via the first fluid resistor 21, the pressing cuff 71, and the on-off valve 16, and exhausted to the atmosphere via the second fluid resistor 22. As a result, even the use of the first fluid resistor 21 and the second fluid resistor 22, a decrease in exhaust efficiency of the sensing cuff 73 can be suppressed as much as possible. Additionally, since the first fluid resistor 21 is disposed on the secondary side of the pressing cuff 71, the pressing cuff 71, which compresses the living body more, is preferentially exhausted, and when the pressure of the pressing cuff 71 falls below that of the sensing cuff 73, an exhaust speed of the sensing cuff 73 increases. Thus, the blood pressure measurement device 1 can reduce a load on the living body due to the compression of the living body after the end of blood pressure measurement.

As described above, according to the blood pressure measurement device 1 according to the first embodiment, the first fluid resistor 21 and the second fluid resistor 22 are provided on the secondary side of the pressing cuff 71, the sensing cuff 73 is connected to the flow path between the first fluid resistor 21 and the second fluid resistor 22, and the secondary side of the second fluid resistor 22 is connected to the atmosphere. Thus, the blood pressure measurement device 1 allows making the pressure ratio between the pressing cuff 71 and the sensing cuff 73 constant.

### Second Embodiment

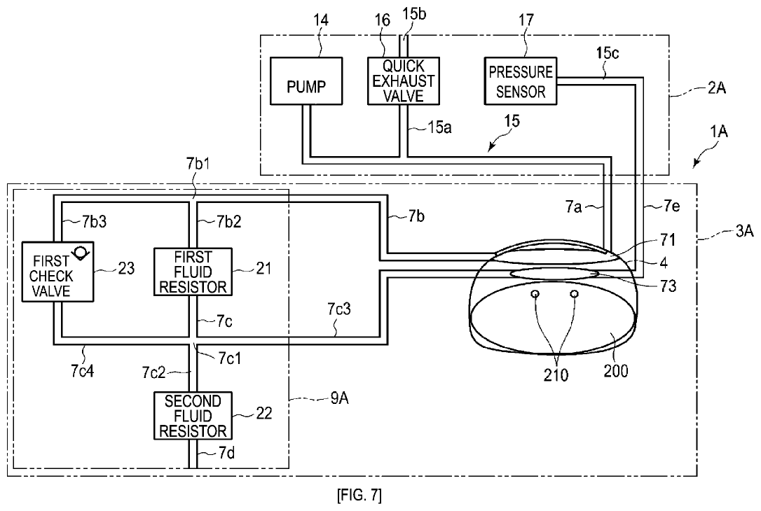

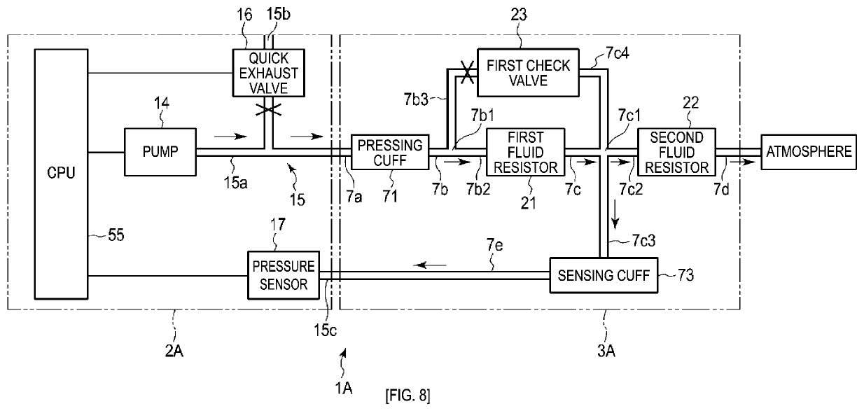

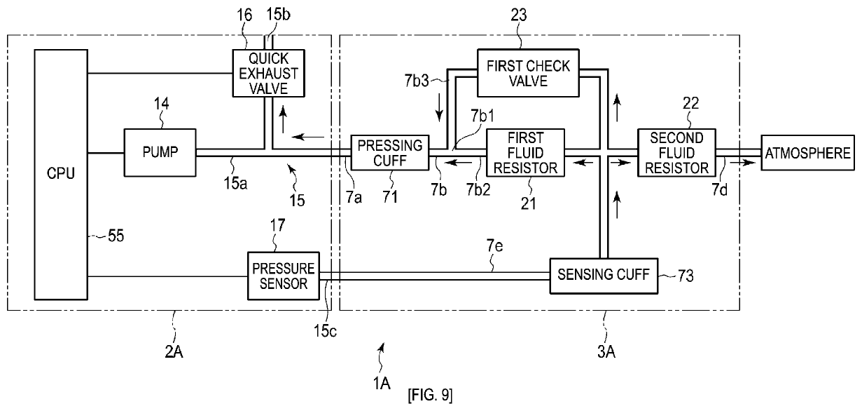

Next, a blood pressure measurement device 1A according to the second embodiment will be described below with reference to FIG. 7 to FIG. 9. FIG. 7 is an explanatory view schematically illustrating a configuration of the blood pressure measurement device 1A according to the second embodiment. FIG. 8 is a block diagram illustrating the configuration of the blood pressure measurement device 1A and illustrating an example of a flow of a fluid supplied to each of the cuffs 71 and 73 in blood pressure measurement. FIG. 9 is a block diagram illustrating the configuration of the blood pressure measurement device 1A and illustrating an example of a flow of a fluid in exhaust of the fluid after blood pressure measurement. Note that in the blood pressure measurement device 1A according to the second embodiment, the same reference signs are given to configurations similar to those of the blood pressure measurement device 1 according to the first embodiment and detailed descriptions thereof are omitted.

Similar to the blood pressure measurement device 1, the blood pressure measurement device 1A is an electronic blood pressure measurement device attached to the living body 200. As illustrated in FIG. 7 to FIG. 9, the blood pressure measurement device 1A includes a device body 2A and a fluid circuit 3A.

The fluid circuit 3A includes the cuff structure 6, the tube group 7, and a fluid control unit 9A. The fluid control unit 9A includes the first fluid resistor 21 and the second fluid resistor 22 connected in series, and a valve 23 provided in parallel with the first fluid resistor 21.

The valve 23 opens when the pressure on the primary side is lower than the pressure on the secondary side. Specifically, the valve 23 closes when the pressure on the pressing cuff 71 side is equal to or more than the pressure on the flow path (the sensing cuff 73) side between the first fluid resistor 21 and the second fluid resistor 22 and opens when the pressure on the pressing cuff 71 side is lower than the pressure on the flow path side between the first fluid resistor 21 and the second fluid resistor 22. The valve 23 is always closed when air is supplied to the pressing cuff 71 and the sensing cuff 73 during blood pressure measurement, for example. The valve 23 opens when the pressure of the pressing cuff 71 falls below the pressure of the sensing cuff 73. The valve 23 is, for example, a check valve. In each of the drawings, the valve 23 is illustrated as the check valve 23.

For example, a cracking pressure of the valve 23 is set to a preferred pressure for exhaust of the pressing cuff 71 and the sensing cuff 73. As a specific example, the cracking pressure of the valve 23 is set to 0 mm Hg such that the valve 23 opens when the pressure of the pressing cuff 71 falls below the pressure of the sensing cuff 73.

Note that the valve 23 is configured to prevent the air in the pressing cuff 71 from flowing toward the sensing cuff 73 side during exhaust, and open when the pressure on the primary side is lower than the pressure on the secondary side. However, when the air does not substantially flow from the pressing cuff 71 to the sensing cuff 73 in exhaust of the fluid circuit 3A, the valve 23 may be set to have the cracking pressure (for example, 15 mm Hg) in which the valve 23 opens when the pressure on the primary side is slightly lower than the pressure on the secondary side.

Note that to connect the valve 23 in parallel with the first fluid resistor 21, for example, the second tube 7b has a branch portion 7b1 in the middle portion from the primary side toward the secondary side, and is a branch pipe branched into two flow paths at the branch portion 7b1. The primary side of the second tube 7b is fluidly connected to the pressing cuff 71. One branched tube portion 7b2 on the secondary side of the second tube 7b is connected to the first fluid resistor 21. The other branched tube portion 7b3 on the secondary side of the second tube 7b is connected to the valve 23.

The third tube 7c has the branch portion 7c1 in the middle portion from the primary side toward the secondary side, and is a branch pipe branched into three flow paths at the branch portion 7c1. The primary side of the third tube 7c is fluidly connected to the first fluid resistor 21. The first tube portion 7c2 on the secondary side of the third tube 7c and branched into three is connected to the second fluid resistor 22. The second tube portion 7c3 on the secondary side of the third tube 7c and branched into three is connected to the sensing cuff 73. The third tube portion 7c4 on the secondary side of the third tube 7c and branched into three is connected to the secondary side of the valve 23.

In the fluid circuit 3A, the on-off valve 16 is closed by the measurement processing unit 55a in the control unit 55 during blood pressure measurement, and when the pump 14 starts driving, the air is first supplied to the pressing cuff 71. Since the air is supplied to the pressing cuff 71 first, the pressure on the primary side of the valve 23 becomes higher than the pressure on the secondary side, and the valve 23 closes. A portion of the air supplied to the pressing cuff 71 passes through the first fluid resistor 21 and is supplied to the flow path between the first fluid resistor 21 and the second fluid resistor 22. At this time, a portion of the air supplied to the pressing cuff 71 according to the resistance of the first fluid resistor 21 is supplied to the flow path between the first fluid resistor 21 and the second fluid resistor 22.

A portion of the air supplied to the flow path between the first fluid resistor 21 and the second fluid resistor 22 is injected into the sensing cuff 73, and the other air supplied to the flow path between the first fluid resistor 21 and the second fluid resistor 22 passes through the second fluid resistor 22 and is exhausted to the atmosphere. At this time, due to the resistance of the second fluid resistor 22, the flow rate of the air flowing to each of the sensing cuff 73 and the atmosphere changes in the air supplied to the flow path between the first fluid resistor 21 and the second fluid resistor 22.

In addition, since the fluid resistance ratio between the first fluid resistor 21 and the second fluid resistor 22 is set, the pressure ratio between the pressure of the pressing cuff 71 and the pressure of the sensing cuff 73 is constant from the start of supply of the air by the pump 14 until the stop of supply of the air.

Further, in the fluid circuit 3A, when the exhaust of the fluid circuit 3A starts after the blood pressure measurement, the pump 14 is stopped by the measurement processing unit 55a in the control unit 55, the on-off valve 16 opens, and the on-off valve 16 side of the pressing cuff 71 is connected to the atmosphere, and thus the air in the pressing cuff 71 flows to the on-off valve 16 side. The air in the sensing cuff 73 is exhausted to the atmosphere via the second fluid resistor 22.

When the exhaust of the pressing cuff 71 progresses and the pressure of the pressing cuff 71 is lower than the pressure of the sensing cuff 73, in addition to the exhaust to the atmosphere via the second fluid resistor 22, the air in the sensing cuff 73 moves to the pressing cuff 71 through the first fluid resistor 21 and is exhausted from the on-off valve 16 to the atmosphere.

In addition, the valve 23 opens, the flow path through the valve 23 becomes a bypass path, and an exhaust speed of the sensing cuff 73 increases. Then, the exhaust of the pressing cuff 71 and the sensing cuff 73 proceeds, and the pressures of the pressing cuff 71 and the sensing cuff 73 become atmospheric pressures. In this way, in the exhaust of the fluid circuit 3A, the pressing cuff 71 is preferentially and quickly exhausted than the sensing cuff 73. In the fluid circuit 3A, when the pressure of the pressing cuff 71 falls below the pressure of the sensing cuff 73, the valve 23 opens, and the pressing cuff 71 and the sensing cuff 73 are quickly exhausted. As such, the fluid circuit 3A is exhausted.

The blood pressure measurement device 1A thus configured produces effects similar to the effects of the blood pressure measurement device 1 according to the first embodiment described above. In addition, by providing the valve 23 in parallel with the first fluid resistor 21, when the pressure of the sensing cuff 73 is higher than the pressing cuff 71 during exhaust, the valve 23 opens, and the air in the sensing cuff 73 passes through the first valve 23 and is exhausted to the atmosphere via the pressing cuff 71 through the bypass path not passing through the fluid resistor 21 or 22. Thus, even when the two fluid resistors 21 and 22 are provided, the sensing cuff 73 can be exhausted via the bypass path in which the fluid resistor is not disposed. Thus, the blood pressure measurement device 1 including the fluid resistors 21 and 22 also allows preventing the reduction in exhaust speed of the sensing cuff 73.

In particular, the configuration of the fluid control unit 9A is effective when the capacity of the pump 14 is low. That is, in a case where the capacity of the pump 14 is low and the flow to the atmosphere is large, a fluid resistance value of the first fluid resistor 21 and the second fluid resistor 22 (the value of the first fluid resistor+the value of the second fluid resistor) disposed between the pressing cuff 71 and the atmosphere release is set to be large such that the flow rate becomes the predetermined flow rate or less. Therefore, when the air in the sensing cuff 73 passes through the first fluid resistor 21 and the second fluid resistor 22 during exhaust, the flow rate decreases. Therefore, the exhaust speed of the sensing cuff 73 decreases. However, providing the bypass path as in the fluid control unit 9A allows quick exhaust of the sensing cuff 73.

As described above, according to the blood pressure measurement device 1A according to the second embodiment, the first fluid resistor 21 and the second fluid resistor 22 are provided on the secondary side of the pressing cuff 71, the sensing cuff 73 is connected to the flow path between the first fluid resistor 21 and the second fluid resistor 22, and the secondary side of the second fluid resistor 22 is connected to the atmosphere. Thus, the blood pressure measurement device 1A allows making the pressure ratio between the pressing cuff 71 and the sensing cuff 73 constant.

### Third Embodiment

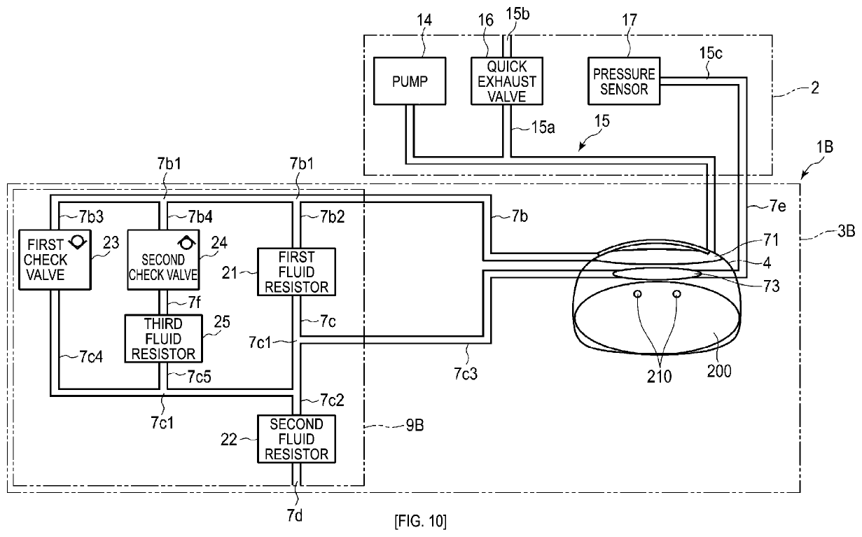

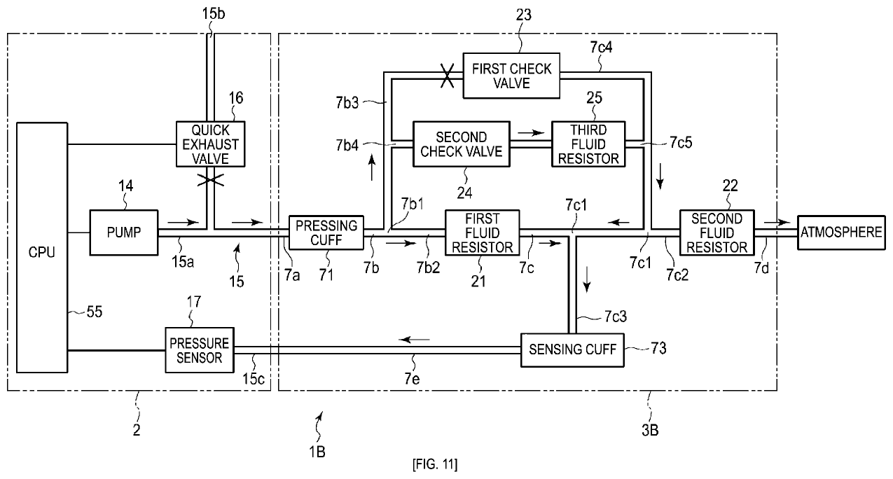

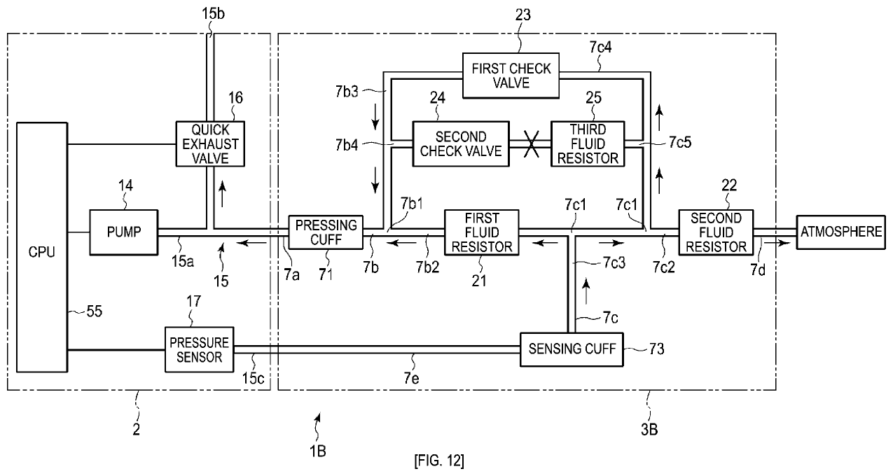

Next, a blood pressure measurement device 1B according to the third embodiment will be described below with reference to FIG. 10 to FIG. 12. FIG. 10 is an explanatory view schematically illustrating a configuration of the blood pressure measurement device 1B according to the third embodiment. FIG. 11 is a block diagram illustrating the configuration of the blood pressure measurement device 1B and illustrating an example of a flow of a fluid supplied to each of the cuffs 71 and 73 in blood pressure measurement. FIG. 12 is a block diagram illustrating the configuration of the blood pressure measurement device 1B and illustrating an example of a flow of a fluid in exhaust of the fluid after blood pressure measurement. Note that in the blood pressure measurement device 1B according to the third embodiment, the same reference signs are given to configurations similar to those of the blood pressure measurement device 1A according to the second embodiment and detailed descriptions thereof are omitted.

Similar to the blood pressure measurement device 1A, the blood pressure measurement device 1B is an electronic blood pressure measurement device attached to the living body 200. As illustrated in FIG. 10 and FIG. 12, the blood pressure measurement device 1B includes the device body 2 and a fluid circuit 3B.

The fluid circuit 3B includes the cuff structure 6, the tube group 7, and a fluid control unit 9B. The fluid control unit 9B includes the first fluid resistor 21 and the second fluid resistor 22 connected in series, the first valve 23 provided in parallel with the first fluid resistor 21, and a second valve 24 and a third fluid resistor 25 connected in parallel with the first fluid resistor 21 and the first valve 23.

The first valve 23 is the valve 23 used in the blood pressure measurement device 1A according to the second embodiment described above.

The second valve 24 opens when the pressure on the primary side is higher than the pressure on the secondary side. Specifically, the second valve 24 opens when the pressure on the pressing cuff 71 side becomes higher than the pressure on the sensing cuff 73 side by equal to or more than a predetermined pressure. The second valve 24 opens when, for example, the pressure of the pressing cuff 71 has a cracking pressure equal to or more than the pressure of the sensing cuff 73 by the predetermined pressure. The second valve 24 is, for example, a check valve.

The third fluid resistor 25 provides the resistance of the passing air. The third fluid resistor 25 has, for example, a flow path cross-sectional area smaller than flow path cross-sectional areas on the primary side and the secondary side of the third fluid resistor 25, that is, flow path cross-sectional areas of the sixth tube 7f and a tube portion 7c5 of the third tube 7c. The third fluid resistor 25 is, for example, orifices.

The cracking pressure of the second valve 24 and the fluid resistance value of the third fluid resistor 25 are set to the values such that the pressure ratio between the pressing cuff 71 and the sensing cuff 73 can be controlled and the pressure of the sensing cuff 73 can be set to the predetermined pressure quickly during blood pressure measurement.

Note that to connect the second valve 24 and the third fluid resistor 25 in parallel with the first fluid resistor 21, for example, the second tube 7b has the branch portion 7b1 in the middle portion from the primary side toward the secondary side, and is a branch pipe branched into three flow paths at the branch portion 7b1.

The primary side of the second tube 7b is fluidly connected to the pressing cuff 71. To the three branched tube portions 7b2, 7b3, 7b4 as the secondary side of the second tube 7b, the first fluid resistor 21, the first valve 23, and the second valve 24 are connected, respectively.

The third tube 7c has the branch portion 7c1 in the middle portion from the primary side toward the secondary side, and is a branch pipe branched into four flow paths at the branch portion 7c1. The primary side of the third tube 7c is fluidly connected to the first fluid resistor 21. Tube portions 7c2, 7c3, 7c4, and 7c5 branched into four on the secondary side of the third tube 7c are connected to the second fluid resistor 22, the sensing cuff 73, the secondary side of the first valve 23, and the third fluid resistor 25, respectively. For example, the tube group 7 includes a sixth tube 7f that connects the second valve 24 and the third fluid resistor 25.

In the fluid circuit 3B, the on-off valve 16 is closed by the measurement processing unit 55a in the control unit 55 during blood pressure measurement, and when the pump 14 starts driving, the air is first supplied to the pressing cuff 71. Since the air is supplied to the pressing cuff 71 first, the first valve 23 closes when the pressure on the primary side of the first valve 23 becomes higher than the pressure on the secondary side. The second valve 24 opens when the pressure on the primary side becomes higher than the pressure on the secondary side by a predetermined value (for example, 150 mm Hg) or more. A portion of the air supplied to the pressing cuff 71 passes through the first fluid resistor 21 and the third fluid resistor 25 and is supplied to a flow path between the first fluid resistor 21, the third fluid resistor 25, and the second fluid resistor 22. At this time, a portion of the air supplied to the pressing cuff 71 according to the resistance values of the first fluid resistor 21 and the third fluid resistor 25 is supplied to the flow path between the first fluid resistor 21, the third fluid resistor 25, and the second fluid resistor 22.

A portion of the air supplied to the flow path between the first fluid resistor 21, the third fluid resistor 25, and the second fluid resistor 22 is injected into the sensing cuff 73, and the other air supplied to the flow path between the first fluid resistor 21, the third fluid resistor 25, and the second fluid resistor 22 passes through the second fluid resistor 22 and is exhausted to the atmosphere. At this time, due to the resistance of the second fluid resistor 22, the flow rate of the air flowing to each of the sensing cuff 73 and the atmosphere changes in the air supplied to the flow path between the first fluid resistor 21, the third fluid resistor 25, and the second fluid resistor 22.

In addition, since the fluid resistance ratio between the first fluid resistor 21, the third fluid resistor 25, and the second fluid resistor 22 is set, the pressures of the pressing cuff 71 and the sensing cuff 73 increase with the constant pressure ratio between the pressure of the pressing cuff 71 and the pressure of the sensing cuff 73 from the start of the supply of air by the pump 14.

Further, in the fluid circuit 3B, when the exhaust of the fluid circuit 3B starts after the blood pressure measurement, the pump 14 is stopped by the measurement processing unit 55a in the control unit 55, the on-off valve 16 opens, and the on-off valve 16 side of the pressing cuff 71 is connected to the atmosphere, and thus the air in the pressing cuff 71 flows to the on-off valve 16 side. The air in the sensing cuff 73 is exhausted to the atmosphere via the second fluid resistor 22. When the exhaust of the pressing cuff 71 progresses, the pressure of the pressing cuff 71 decreases, and when the difference in pressure with the sensing cuff 73 becomes the predetermined value or less, the second valve 24 closes and, in addition to the exhaust to the atmosphere via the second fluid resistor 22, the air in the sensing cuff 73 moves to the pressing cuff 71 through the first fluid resistor 21 and is exhausted from the on-off valve 16 to the atmosphere. In addition, when the pressure of the pressing cuff 71 is lower than the pressure of the sensing cuff 73, the first valve 23 opens, and the air in the sensing cuff 73 moves to the pressing cuff 71 via the first valve 23. Thus, the fluid circuit 3B is exhausted.

The blood pressure measurement device 1B thus configured produces effects similar to the effects of the blood pressure measurement device 1A according to the second embodiment described above. In addition, the fluid control unit 9B includes the second valve 24 and the third fluid resistor 25 disposed in series and in parallel with the first fluid resistor 21. As a result, the blood pressure measurement device 1B can reduce the differential pressure between the pressing cuff 71 and the sensing cuff 73 and can improve a climbing speed of the pressure of the sensing cuff 73 to the predetermined pressure during blood pressure measurement. Furthermore, the reduction in capacity of the pump 14 is allowed.

Specifically, to control the pressure ratio, as the pressure of the pressing cuff 71 increases, the differential pressure with the pressure of the sensing cuff 73 increases, and therefore a pump having a higher capacity is required. However, the blood pressure measurement device 1B can reduce the differential pressure between the pressing cuff 71 and the sensing cuff 73 by the second valve 24 and the third fluid resistor 25 provided in parallel with the first fluid resistor 21. Thus, the blood pressure measurement device 1B can shorten the time taken for increasing the pressure of the sensing cuff 73 to the pressure required for blood pressure measurement without the need for the pump 14 having the high capacity.

### Fourth Embodiment

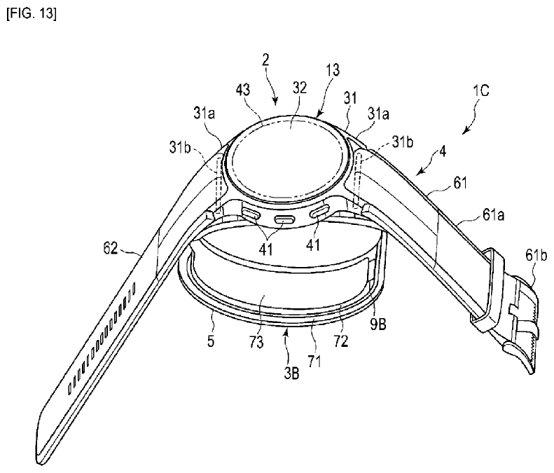

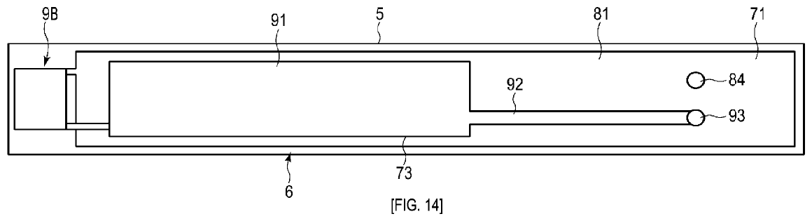

Next, a configuration of a blood pressure measurement device 1C according to the fourth embodiment will be described below with reference to FIG. 13 and FIG. 14. Note that the blood pressure measurement device 1C according to the fourth embodiment is an example in which the blood pressure measurement device 1B according to the third embodiment described above is applied to a wearable blood pressure measurement device attached to the wrist 200 as a living body. In the configuration of the blood pressure measurement device 1C according to the fourth embodiment, the same reference signs are given to configurations similar to those of the blood pressure measurement device 1B according to the third embodiment described above and detailed descriptions thereof are omitted.

As illustrated in FIG. 13, the blood pressure measurement device 1C includes the device body 2, the fluid circuit 3B, the belt 4, which is a fixture that fixes the device body 2 to the wrist, and a curler 5 disposed between the belt 4 and the wrist 200.

As illustrated in FIG. 13, the case 11 of the device body 2 includes an outer case 31 and a windshield 32 covering an opening on the side opposite (outer side) from the wrist 200 side of the outer case 31. Additionally, the case 11 includes a rear cover provided on the wrist 200 side inside the outer case 31.

The outer case 31 is formed in a cylindrical shape. The outer case 31 includes pairs of lugs 31a provided at respective symmetrical positions in the circumferential direction of an outer circumferential surface, and spring rods 31b each provided between each of the two pairs of lugs 31a. The windshield 32 is, for example, a circular glass plate. Additionally, a base portion that supports each of the components is provided inside the outer case 31.

The display device 12 is disposed on the base portion of the outer case 31 and directly below the windshield 32.

The operation device 13 is configured to allow the user to input an instruction. As illustrated in FIG. 13, for example, the operation device 13 includes a plurality of buttons 41 provided on the case 11, a sensor that detects the operation of the buttons 41, and a touch panel 43 provided on the display device 12 or the windshield 32. When operated by the user, the operation device 13 converts an instruction into an electrical signal. The sensor and the touch panel 43 are electrically connected to the control substrate 20 to output electrical signals to the control substrate 20.

As illustrated in FIG. 13, the belt 4 includes a first belt 61 provided on one of the pair of lugs 31a and the spring rod 31b, a second belt 62 provided on the other pair of lugs 31a and the spring rod 31b, and a connector that connects the first belt 61 and the second belt 62. The belt 4 is wrapped around the wrist 200 with the curler 5 in between. Note that in the present embodiment, the connector is a buckle 61b provided to the first belt 61.

The first belt 61 is referred to as a so-called a parent and is configured like a band capable of being joined to the second belt 62. As illustrated in FIG. 13, the first belt 61 includes a belt portion 61a and the buckle 61b. The belt portion 61a is configured like a band. The belt portion 61a is formed of an elastically deformable resin material. In addition, the belt portion 61a is flexible and includes a sheet-like insert member inside the belt portion 61a for suppressing stretching in the longitudinal direction of the belt portion 61a.

The belt portion 61a is provided with the spring rod 31b at one end portion and the buckle 61b at the other end portion. The spring rod 31b provided on the one end portion of the first belt 61 is disposed between the pair of lugs 31a, and thus the first belt 61 is rotatably held to the outer case 31.

The second belt 62 is referred to as so-called tip of a blade and formed like a band. The second belt 62 is formed of an elastically deformable resin material. In addition, the second belt 62, for example, is flexible and includes a sheet-like insert member inside the second belt 62 for suppressing stretching in the longitudinal direction of the second belt 62.

The second belt 62 is fixed to the buckle 61b. The second belt 62 is provided with the spring rod 31b at one end portion. The spring rod 31b provided on the one end portion of the second belt 62 is disposed between the pair of lugs 31a, and thus the second belt 62 is rotatably held to the outer case 31.

Thus, the first belt 61 and the second belt 62 of the belt 4 are integrally connected together by the buckle 61b, and the belt 4 comes to have an annular shape following along the circumferential direction of the wrist 200 along with the outer case 31. By shaping the belt 4 in an annular shape following along the circumferential direction of the wrist, the curler 5 is pressed and elastically deformed to follow along the circumferential direction of the wrist of the wearer of the blood pressure measurement device 1C.

As illustrated in FIG. 13, the curler 5 is configured in a band-like shape that curves in such a manner as to follow along the circumferential direction of the wrist 200. The curler 5 is formed with a first end and a second end spaced apart from each other. For example, the outer surface on the first end side of the curler 5 is fixed to the rear cover side of the device body 2 or is integrally formed with the rear cover of the device body 2 and the base portion. The curler 5 is disposed, for example, at a position where the first end and the second end protrude more to one side of the wrist 200 than the device body 2. Accordingly, the curler 5 is disposed with the first end and the second end to the side of the wrist 200 when the blood pressure measurement device 1C is attached to the wrist 200. Furthermore, the first end and the second end of the curler 5 are located adjacent to each other at a predetermined distance from each other. The curler 5 is formed of a resin material, for example.

The curler 5 with such a configuration is fixed to the outer case 31 with the first end and the second end orientated to face the second belt 62 of the belt 4. Also, the curler 5 at least at the position facing the hand palm-side of the wrist 200 curves along the circumferential direction along with the hand palm-side of the wrist 200, and thus the cuff structure 6 facing the hand palm-side of the wrist 200 is held in a curved state following along the shape of the hand palm-side of the wrist 200.

The curler 5 has a hardness appropriate to provide flexibility and shape retainability. Here, “flexibility” refers to deformation of the shape of the curler 5 in a radial direction at the time of application of an external force of the belt 4 to the curler 5. For example, “flexibility” refers to deformation of the shape of the curler 5 in a side view in which the curler 5 approaches the wrist, is along the shape of the wrist, or follows to the shape of the wrist when the curler 5 is pressed by the belt 4. Furthermore, “shape retainability” refers to the ability of the curler 5 to maintain a pre-imparted shape when no external force is applied to the curler 5. For example, “shape retainability” refers to, in the present embodiment, the ability of the curler 5 to maintain the shape in a shape curving along the circumferential direction of the wrist. In the curler 5, the cuff structure 6 is disposed on the inner circumferential surface.

For example, in the fluid circuit 3B, the cuff structure 6, the tube group 7, and the fluid control unit 9B are integrally formed. For example, the fluid circuit 3B is configured by integrally incorporating the tube group 7 and the fluid control unit 9B into a portion of the cuff structure 6.

As a specific example, as illustrated in FIG. 13 and FIG. 14, the cuff structure 6 includes the pressing cuff 71, the sensing cuff 73, and the fluid control unit 9B. FIG. 14 is a developed view illustrating the configuration of the curler 5 and the cuff structure 6. In the cuff structure 6, the pressing cuff 71 and the sensing cuff 73 are stacked and fixed in this order from the inner peripheral surface of the curler 5 to the wrist side, on the inner peripheral surface on the hand palm side of the wrist of the curler 5.

The cuff structure 6 includes a back plate 72 that supports the sensing cuff 73 between the pressing cuff 71 and the sensing cuff 73, for example. As a specific example, the back plate 72 is formed to have a length so as to cover the hand palm side of the wrist 200. The back plate 72 transmits the pressing force from the pressing cuff 71 to the main surface on the back plate 72 side of the sensing cuff 73 in a state in which the back plate 72 runs along the shape of the wrist.

The pressing cuff 71 is set to, for example, substantially the same length as the length in the longitudinal direction of the curler 5. The pressing cuff 71 includes a plurality of, for example, two-layer air bags 81 and a connection portion 84 provided on the first end side in the longitudinal direction. The pressing cuff 71 is provided with the fluid control unit 9B on the second end side in the longitudinal direction.

Here, the air bag 81 has a bag-like structure. The plurality of air bags 81 are stacked and are in fluid communication with one another in the stacking direction. Each of the air bags 81 is formed in a rectangular bag-like shape that is long in one direction. Additionally, the air bag 81 is set such that the width in the lateral direction is the same as or slightly smaller than the width in the lateral direction of the curler 5. The air bag 81 is formed by, for example, combining two sheet members and thermally welding the sheet members in a rectangular frame shape long in one direction using heat. In addition, the two-layer air bags 81 are formed by integrally combining the two air bags 81 by welding using heat, or welding the facing sheet members of the adjacent air bags 81 and after that welding the air bags 81.

The connection portion 84 is, for example, a nipple. The connection portion 84 protrudes from the air bag 81. The connection portion 84 is the first tube 7a connected to the flow path unit 15.

The sensing cuff 73 is, for example, set to have a length such that the sensing cuff 73 can be disposed in a region where the artery is present in the wrist. The sensing cuff 73 faces the region where the artery is present in the wrist with the blood pressure measurement device 1B attached to the wrist. The artery as used herein is a radial artery and/or an ulnar artery. The sensing cuff 73 is inflated to compress the region where the artery on the hand palm side is present in the wrist. The sensing cuff 73 is pressed by the inflated pressing cuff 71 to the wrist side.

As a specific example, the sensing cuff 73 includes one air bag 91, a flow path body 92 that communicates with the air bag 91, and a connection portion 93 provided at the leading end of the flow path body 92. The sensing cuff 73 is constituted by integrally welding two sheet members.

The air bag 91 is constituted in a rectangular shape that is long in one direction. The air bag 91 is, for example, set to have a length such that the air bag 91 can be disposed in a region where the artery is present in the wrist. The air bag 91 is formed by, for example, combining two sheet members long in one direction and thermally welding the sheet members in a rectangular frame shape long in one direction using heat.

The flow path body 92 is integrally provided at a portion of one edge portion of the air bag 91 in the longitudinal direction. As a specific example, the flow path body 92 is provided at the end portion of the air bag 91 near the device body 2. Additionally, the flow path body 92 is, for example, formed in a shape that is long in one direction, has a width smaller than the width of the air bag 91 in the lateral direction, and is formed with a leading end having a circular shape. The flow path body 92 includes the connection portion 93 on the leading end.

The flow path body 92 is constituted by welding the two sheet members in a frame shape long in one direction using heat, in a state where the connection portion 93 is disposed on the two sheet members. Note that, portions of the weld portions where the two sheet members are welded in a rectangular frame shape are not welded and the air bag 91 is constituted to be continuous with a weld portion forming the flow path body 92, and thus the air bag 91 fluidly continues with the flow path body 92.

The connection portion 93 is, for example, a nipple. The connection portion 93 is provided at the leading end of the flow path body 92. Also, the leading end of the connection portion 93 is externally exposed from the sheet member on the side of facing the curler 5 among the two sheet members constituting the flow path body 92. The connection portion 93 is connected to the flow path unit 15.

Thus, the flow path body 92 and the connection portion 93 are connected to the flow path unit 15 via the connection portion 93, and constitute the fifth tube 7e that connects the air bag 91 and the pressure sensor 17.

In the cuff structure 6 configured in this manner, the pressing cuff 71 includes the first tube 7a of the tube group 7 and the sensing cuff 73 includes the fifth tube 7e.

The fluid control unit 9B is disposed, for example, on an inner surface of the curler 5 and at an end portion on the hand palm side of the wrist. The fluid control unit 9B is integrally formed with the end portions of the pressing cuff 71 and the sensing cuff 73. As a specific example, the fluid control unit 9B is integrally formed with the end portion of the pressing cuff 71 and a portion of which is fluidly connected to the sensing cuff 73.

For example, the fluid control unit 9B includes the second tube 7b, the third tube 7c, the fourth tube 7d, the first fluid resistor 21, the second fluid resistor 22, the first valve 23, the second valve 24, and the third fluid resistor 25. In the fluid control unit 9B, the second tube 7b, the third tube 7c, the fourth tube 7d, the first fluid resistor 21, the second fluid resistor 22, the first valve 23, the second valve 24, and the third fluid resistor 25 are integrally formed.

The second tube 7b, the third tube 7c, and the fourth tube 7d are formed by, for example, a portion of a pair of the seat members constituting one air bag 81 of the pressing cuff 71. For example, the second tube 7b, the third tube 7c, and the fourth tube 7d are clearances formed between the pair of seat members by not welding the regions constituting the second tube 7b, the third tube 7c, and the fourth tube 7d when the pair of sheet members are welded. The first fluid resistor 21, the second fluid resistor 22, the first valve 23, the second valve 24, and the third fluid resistor 25 are disposed in the clearances between the pair of sheet members constituting the second tube 7b, the third tube 7c, and the fourth tube 7d. Further, the tube portion 7c3 on the secondary side of the branch portion 7c1 of the third tube 7c is connected to the sensing cuff 73.

The blood pressure measurement device 1C thus configured produces effects similar to the effects of the blood pressure measurement device 1B according to the third embodiment described above. In addition, in the blood pressure measurement device 1C, the second tube 7b, the third tube 7c, the fourth tube 7d, the first fluid resistor 21, the second fluid resistor 22, the first valve 23, the second valve 24, and the third fluid resistor 25 are integrally formed to form the fluid control unit 9B, and the fluid control unit 9B is integrally connected to the end portions of the pressing cuff 71 and the sensing cuff 73. The fluid control unit 9B is configured to be disposed at the end portion of the curler 5. In the blood pressure measurement device 1C, the fluid control unit 9B can be disposed integrally with the pressing cuff 71 and the sensing cuff 73 on the curler 5. This eliminates the need for disposing the fluid control unit 9B in the device body 2 and allows miniaturizing the device body 2. In addition, since the fluid control unit 9B is disposed on the end portion of the curler 5, it is possible to prevent the fluid control unit 9B from inhibiting blood pressure measurement during blood pressure measurement.

### Other Embodiments

Note that the present invention is not limited to the embodiments described above. For example, in the examples described above, in the blood pressure measurement device 1, 1A, 1B, or 1C according to each of the embodiments, an example in which the fluid circuit 3, 3A, or 3B is disposed outside the device body 2 has been described, but the configuration is not limited to this. For example, the blood pressure measurement device 1, 1A, or 1B may be configured to house a portion of the configuration of the fluid circuit 3, 3A, or 3B in the device body 2.

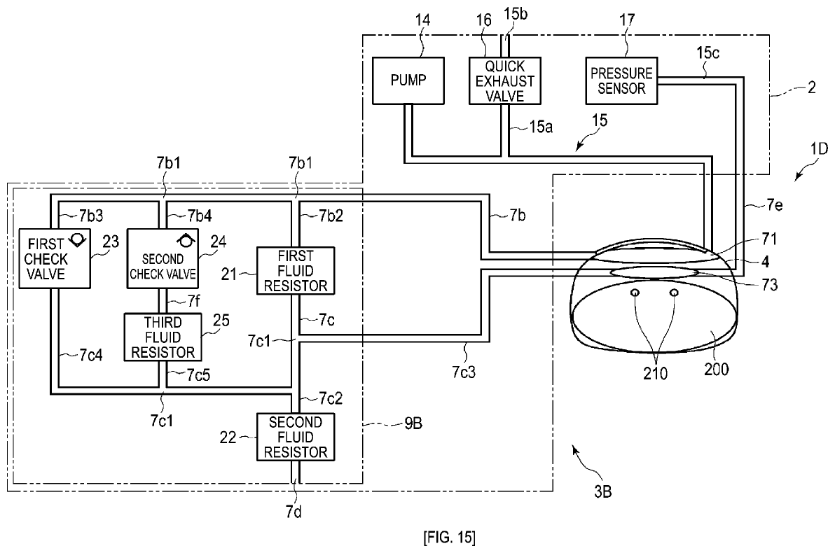

As a specific example, like a blood pressure measurement device 1D according to a fifth embodiment illustrated in FIG. 15, for example, among the configurations of the fluid circuit 3B, the first fluid resistor 21, the second fluid resistor 22, the first valve 23, the second valve 24, and the third fluid resistor 25 constituting the fluid control unit 9B and a portion of the tube group 7 for fluidly connecting them to another configuration may be housed in the device body 2. Further, similarly, the fluid control unit 9 or 9A may also be housed in the device body 2.

In addition, in the example described above, as an example of applying the blood pressure measurement device 1B according to the third embodiment to the wearable blood pressure measurement device attached to the wrist 200, the blood pressure measurement device 1C according to the fourth embodiment is described, but the configuration is not limited thereto. For example, the blood pressure measurement device 1 according to the first embodiment and the blood pressure measurement device 1A according to the second embodiment may be applied to a wearable blood pressure measurement device equivalent to the wearable blood pressure measurement device 1C according to the fourth embodiment.

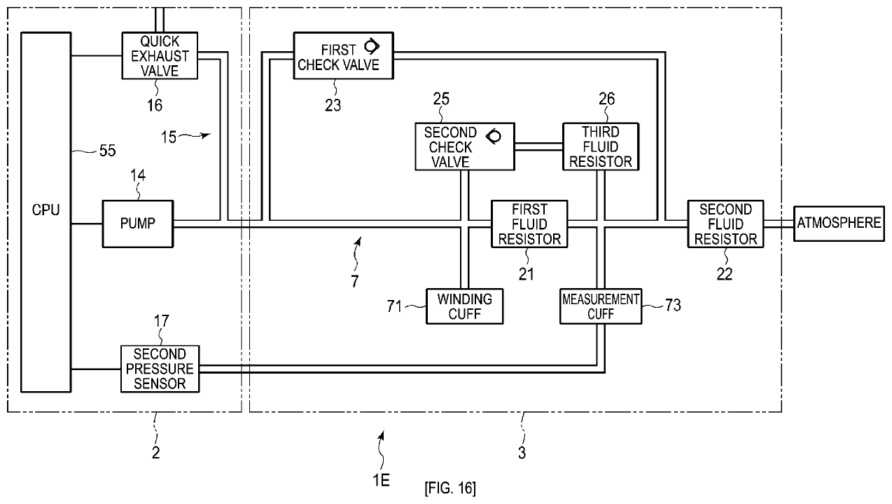

The blood pressure measurement device 1, 1A, or 1B may be configured to be attached to an upper arm. In such a configuration, in the blood pressure measurement device 1, 1A, or 1B, the first cuff 71 may be used as the winding cuff 71 that is wound around the upper arm and the second cuff 73 may be used as the measurement cuff 73. For example, FIG. 16 illustrates an example of a blood pressure measurement device 1E attached to the upper arm according to a sixth embodiment. Note that similarly, in the blood pressure measurement device 1E attached to the upper arm as well, the first cuff 71 may be used as the winding cuff In addition, in the case of the blood pressure measurement device 1 or 1A attached to the upper arm, an automatic winding function may be provided.

Additionally, the blood pressure measurement device 1, 1A, or 1B described above has been described with a reduced pressure measurement method as an example of blood pressure measurement, but the method is not limited thereto. Each of the blood pressure measurement devices 1, 1A, and 1B may employ a pressurization measurement method as an example of blood pressure measurement. In the case of the blood pressure measurement device 1, 1A, or 1B of the pressure measurement method, as a blood pressure measurement device according to a seventh embodiment, the on-off valve 16 may be a quick exhaust valve that allows quick exhaust and the blood pressure may be measured by pressurization measurement method.

Additionally, for example, in the examples described above, the configuration in which the blood pressure measurement device 1, 1A, 1B, 1C, or 1D includes the two cuffs 71 and 73 has been described, but the configuration is not limited to this.

That is, the blood pressure measurement device may be configured to include three or more cuffs.

In the case of the blood pressure measurement device including the plurality of cuffs, for example, a plurality of fluid resistors may be provided in series on the secondary side of the cuff on the most primary side, each of the cuffs may be connected between the adjacent fluid resistors, and the fluid resistor on the most secondary side may be connected to the atmosphere.

An example of the three cuffs will be described using a blood pressure measurement device 1F according to an eighth embodiment illustrated in FIG. 17. The blood pressure measurement device 1F includes, for example, a device body 2 and a fluid circuit 3F. In addition to the pressing cuff 71 and the sensing cuff 73, the fluid circuit 3F can include, for example, a tensile cuff 74 disposed on a hand back side of the wrist and inflating to pull the side of the hand of the wrist as the third cuff 74, and an assist cuff that presses the hand back side of the hand. Note the four or more cuffs may be used.

Also, in the case where the three cuffs 71, 73, and 74 are provided, for example, as illustrated in FIG. 17, the first to third fluid resistors 21, 22, and 26 are connected in series to the secondary side of the tensile cuff 74 and the third fluid resistor 26 is connected to the atmosphere. Also, the pressing cuff 71 is connected between the first fluid resistor 21 and the second fluid resistor 22, and the sensing cuff 73 is connected between the second fluid resistor 22 and the third fluid resistor 26. Further, for example, the first valve 23 is connected to the primary side of the first fluid resistor 21, connected to a flow path between the first fluid resistor 21 and the second fluid resistor 22, and connected to a flow path between the second fluid resistor 22 and the third fluid resistor 26. For example, the second valve 24 and a fourth fluid resistor 25 are connected in parallel with the second fluid resistor 22.

With such a configuration, even when the three cuffs 71, 73, and 74 are provided, the pressure ratio can be controlled to be constant by the use of partial pressures of the fluid resistors 21, 22, and 26.

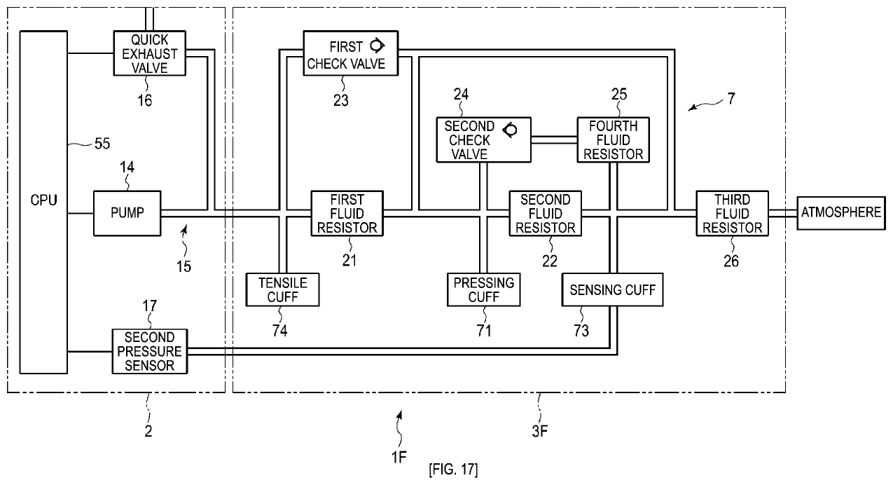

In the configuration in which the plurality of cuffs are provided, the configuration is not limited to the blood pressure measurement device 1F according to the eighth embodiment described above. For example, as in a blood pressure measurement device 1G according to a ninth embodiment illustrated in FIG. 18, the pressing cuff 71 and the sensing cuff 73 may be connected by the fluid control unit 9B and the tensile cuff 74 and the pressing cuff 71 may be connected via a third valve 27 and a fourth fluid resistor 28.

For example, the third valve 27 is a check valve that opens when the pressure of the tensile cuff 74 is higher than the pressure of the pressing cuff 71 and the differential pressure between the tensile cuff 74 and the pressing cuff 71 becomes a predetermined differential pressure. The cracking pressure of the third valve 27 and the resistance value of the fourth fluid resistor 28 are appropriately set by, for example, an inflow amount of air to the secondary side of the tensile cuff 74 and the pressure required for inflation of each of the cuffs 71, 73, and 74.

The blood pressure measurement device 1G causes the tensile cuff 74 to inflate first, and then by inflating the pressing cuff 71 and the sensing cuff 73, it is possible to reduce creases during inflation of each of the cuffs 71, 73, and 74. The blood pressure measurement device 1G may include one of the third valve 27 and the fourth fluid resistor 28.

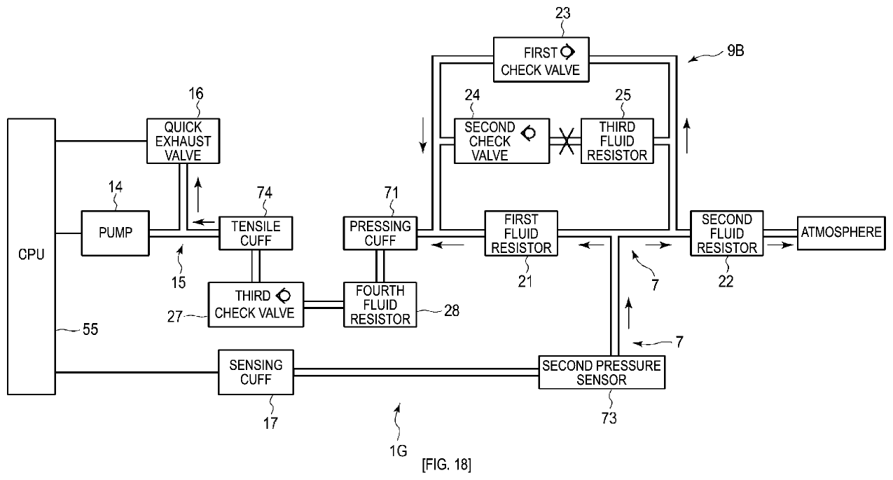

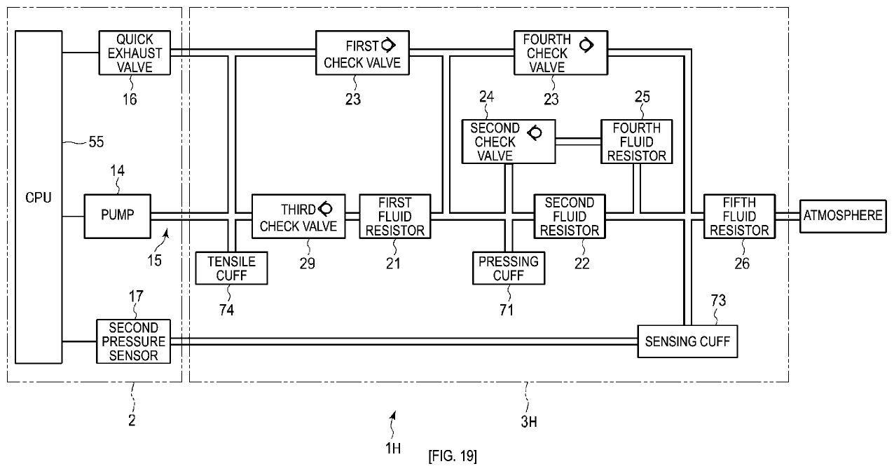

In addition, as another example, as in the fluid circuit 3H of a blood pressure measurement device 1H according to a tenth embodiment illustrated in FIG. 19, the configuration may be similar to the configuration of the blood pressure measurement device 1F according to the eighth embodiment and further a third valve 29 as a check valve may be provided on the primary side of the first fluid resistor 21. For example, as illustrated in FIG. 19, the third valve 29 may have a cracking pressure such that the third valve 29 opens when the differential pressure between the pressure on the secondary side of the tensile cuff 74 and the pressure on the secondary side of the third valve 29 is a predetermined differential pressure or more. In the blood pressure measurement device 1H, when the differential pressure between the pressures on the secondary sides of the tensile cuff 74 and the third valve 29 reaches the predetermined differential pressure, the third valve 29 opens and air is supplied to the pressing cuff 71 and the sensing cuff 73. In addition, the blood pressure measurement device 1H may include the two first valves 23. Note that, as an example of the configuration in which the two first valves 23 are provided, FIG. 19 illustrates the first check valve 23 and a fourth check valve 23.

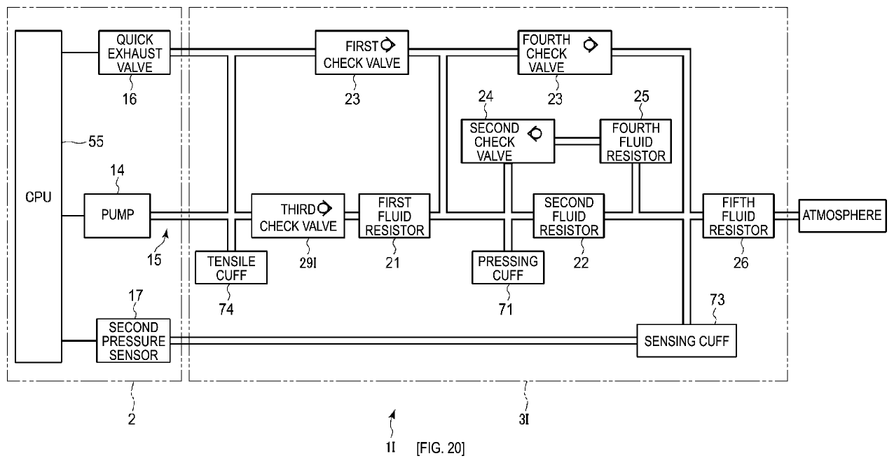

Further, as a modified example of the blood pressure measurement device 1H, as a blood pressure measurement device 1I according to an eleventh embodiment illustrated in FIG. 20, when the differential pressure between the pressures on the secondary sides of the tensile cuff 74 and a third valve 29I reaches the predetermined differential pressure, the third valve 29I closes, and afterward, the supply of air to the pressing cuff 71 and the sensing cuff 73 stops.

In addition, in the example described above, the example in which the two or more fluid resistors 21, 22, 25, and 28 are used as the fluid control units 9, 9A, and 9B that control (differential pressure control) the pressure ratio of the air in the plurality of cuffs and in the atmosphere to be constant by the fluid resistance ratio has been described. Further, the example in which the fluid resistors 21, 22, 25, and 28 are orifices has been described. Here, the orifice is the flow rate resistor that reduces the pressure on the downstream side of the orifice when the fluid flows by reducing the flow path.

Here, the increase in flow rate resistor of the orifice is remarkable as the flow rate resistor (fluid resistor) at low pressure increases due to an influence of viscosity and as the orifice diameter decreases. Therefore, by connecting the orifices having small orifice diameters in parallel, it is possible to obtain the flow rate resistor with high pressure dependence. Also, by connecting the orifices having large diameters in series, the flow rate resistor having low pressure dependence can be obtained. For example, the orifice diameters of the flow rate resistors disposed in series are set to be larger than the orifice diameters of the flow rate resistors disposed in parallel. In this way, setting the orifice diameters and the arrangement of the plurality of flow rate resistors allow configuring the fluid resistors 21, 22, 25, and 28 taking into account a pressure dependent effect.

Thus, at least one of the two or more fluid resistors 21, 22, 25, and 28 used in the blood pressure measurement device 1 may be configured by combining a plurality of flow rate resistors as orifices. That is, the fluid resistors 21, 22, 25, or 28 may have a configuration in which pressure dependence is appropriately set by disposing the plurality of flow rate resistors in series, in parallel, or a combination of in series and parallel.

By providing the plurality of flow rate resistors in series, in parallel, or in series and parallel, the resistance values and the pressure dependence of the fluid resistors 21, 22, 25, and 28 can be arbitrarily set. In other words, by appropriately setting the plurality of flow rate resistors, the fluid control units 9, 9A, and 9B can be strictly matched with required cuff properties. That is, in the differential pressure control of the fluid control units 9, 9A, and 9B, depending on the pressure value in the cuff, a deviation between the differential pressure and the insufficient compression force of the pressing cuff possibly increases. However, by preferably setting the pressure dependence of the fluid resistors 21, 22, 25, and 28, the blood pressure measurement device can set the arbitrary compression properties, such as decreasing or increasing the pressure of compressing the wrist 200 by the cuff structure 6. Therefore, the deviation between the differential pressure and the insufficient compression force of the pressing cuff can be reduced regardless of the pressure value in the cuff, and the accuracy of blood pressure measurement by the blood pressure measurement device can be improved.

Next, as an example of the blood pressure measurement device 1 including the first fluid resistor 21 and the second fluid resistor 22 using the plurality of orifices (flow rate resistors), the blood pressure measurement device 1 according to a twelfth embodiment will be described with reference to FIG. 21 to FIG. 29.

FIG. 21 is an explanatory view illustrating the blood pressure measurement device 1 according to the twelfth embodiment. For example, as illustrated in FIG. 21, the blood pressure measurement device 1 includes the first fluid resistor 21 provided between the pressing cuff 71 and the sensing cuff 73 and the second fluid resistor 22 provided between the sensing cuff 73 and the atmosphere.

For example, the first fluid resistor 21 includes a plurality of, for example, three flow rate resistors 21a, 21b, and 21c disposed in series formed by the orifices. For example, the second fluid resistor 22 includes a plurality of, for example, three flow rate resistors 22a, 22b, and 22c disposed in parallel formed by the orifices. Also, the orifice diameters of the flow rate resistors 21a, 21b, and 21c disposed in series are, for example, set to have diameters larger than orifice diameters of the three flow rate resistors 22a, 22b, 22c disposed in parallel.

According to the blood pressure measurement device 1 configured in this manner, the first fluid resistor 21 in which the three flow rate resistors 21a, 21b, and 21c are connected in series is configured, and thus the flow rate resistor having low pressure dependence can be obtained between the pressing cuff 71 and the sensing cuff 73. In addition, the second fluid resistor 22 in which the three flow rate resistors 22a, 22b, and 22c are connected in parallel is configured, and thus the flow rate resistor having high pressure dependence can be obtained between the sensing cuff 73 and the atmosphere.

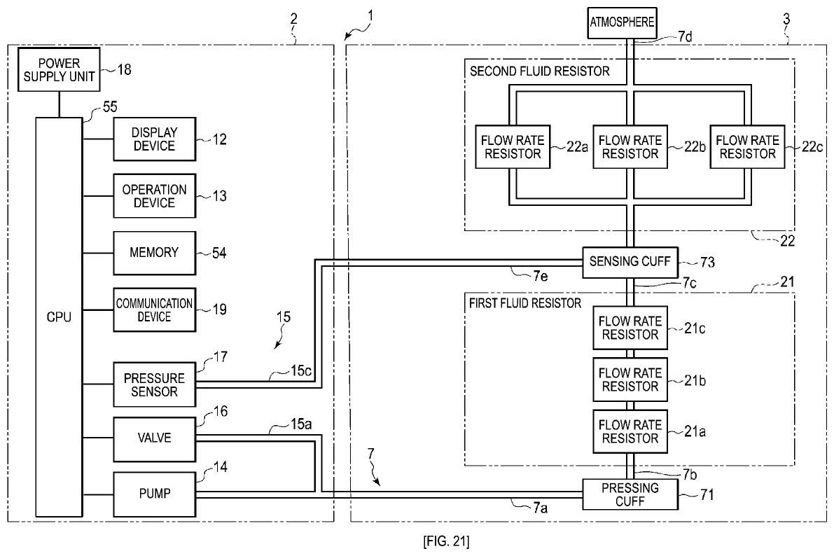

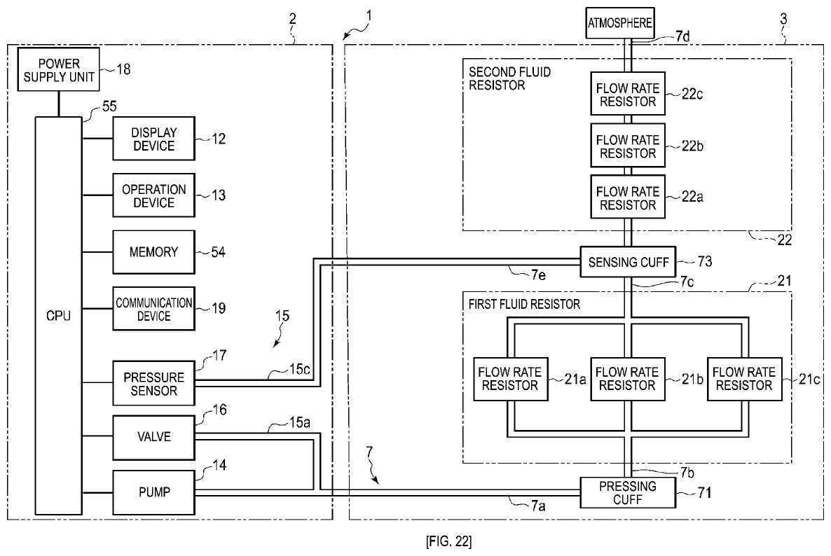

Note that, as in the modified example of the blood pressure measurement device 1 according to the twelfth embodiment illustrated in FIG. 22, the first fluid resistor 21 may have a configuration in which the plurality of flow rate resistors 21a, 21b, and 21c are connected in parallel and the second fluid resistor 22 may have a configuration in which the plurality of flow rate resistors 22a, 22b, 22c are connected in series. In addition, for example, one of the first fluid resistor 21 and the second fluid resistor 22 may be formed by a single fluid resistor (orifice), and the other first fluid resistor 21 or second fluid resistor 22 may be configured to connect a plurality of flow rate resistors in parallel or in series.

Note that each of the first fluid resistor 21 and the second fluid resistor 22 may have a configuration in which a plurality of flow rate resistors are disposed in series or the plurality of flow rate resistors are disposed in parallel.

Note that when the plurality of flow rate resistors are disposed in series, in parallel, or in series and parallel, a plurality of orifices may be combined, but, for example, for miniaturizing the device, an orifice film or an orifice sheet may be used.

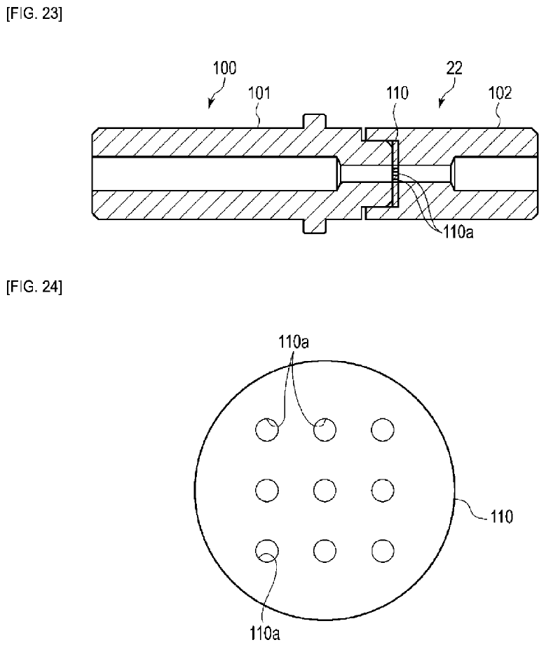

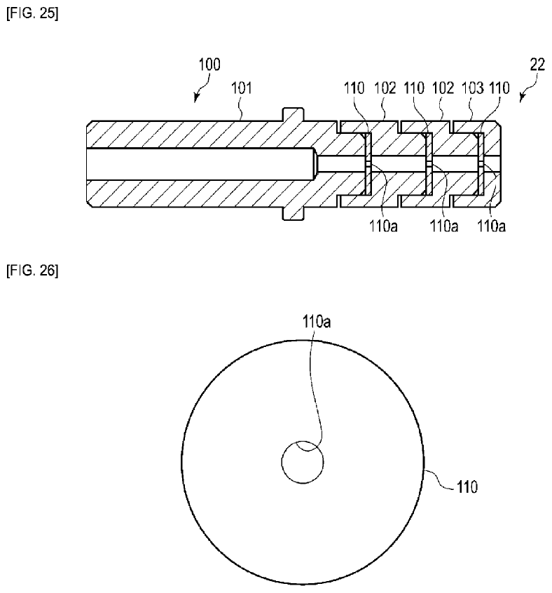

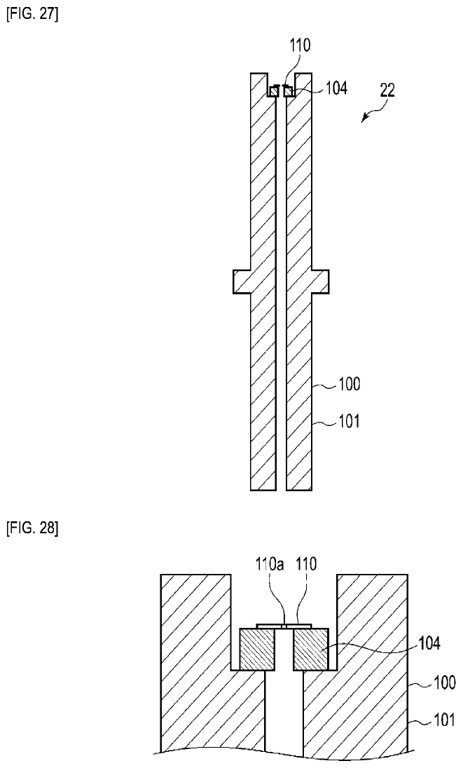

Next, FIG. 23 and FIG. 24 illustrate an example of a fluid resistor 100 in which the flow rate resistors (orifices) are connected in parallel using the orifice film.

For example, the fluid resistor 100 is an example of the second fluid resistor 22 illustrated in FIG. 21 or the first fluid resistor 21 illustrated in FIG. 22, but can be used as appropriate to the fluid resistors 21, 22, 25, and 28 of the respective embodiments described above. The fluid resistor 100 includes a main body 101, a sub-body 102, and an orifice film 110 having a plurality of orifice holes 110a. The main body 101 and the sub-body 102 are fixed by, for example, fitting, thereby crimping and holding the orifice film 110. For example, the main body 101 and the sub-body 102 are set to have diameters such that the leading end of the main body 101 and the rear end of the sub-body 102 are fittable.

For example, the main body 101 and the sub-body 102 are formed in a hollow cylindrical shape so as to ensure forming a flow path. The orifice film 110 is held between the main body 101 and the sub-body 102. Additionally, the orifice film 110 has the plurality of orifice holes 110a in a region where the plurality of orifice holes 110a face the hollow portions of the main body 101 and the sub-body 102. Each of the orifice holes 110a forms the flow rate resistor. Note that the number and the diameter of the orifice holes 110a can be appropriately set.

According to the fluid resistor 100 configured in this manner, the plurality of flow rate resistors (orifices) disposed in parallel can be formed by the plurality of orifice holes 110a formed in the orifice film 110. Thus, the fluid resistor 100 can be miniaturized. The use of the fluid resistor 100 allows miniaturizing the blood pressure measurement device.

Next, FIG. 25 and FIG. 26 illustrate an example of the fluid resistor 100 in which the flow rate resistors (orifices) are connected in series using the orifice film.

For example, the fluid resistor 100 is an example of the second fluid resistor 22 illustrated in FIG. 22 or the first fluid resistor 21 illustrated in FIG. 21, but can be used as appropriate to the fluid resistors 21, 22, 25, and 28 of the respective embodiments described above. The fluid resistor 100 includes the main body 101, the plurality of sub-bodies 102, a cap 103, and the orifice film 110 having the single orifice hole 110a. The main body 101, the plurality of sub-bodies 102, and the cap 103 are stacked and fixed by, for example, fitting, thereby crimping and holding the orifice films 110 therebetween.

For example, the main body 101, the plurality of sub-bodies 102, and the cap 103 are formed in a hollow cylindrical shape so as to ensure forming a flow path. The cap 103, for example, has a leading end portion that is formed in a planar shape and has a diameter in which another member cannot be fixed to the leading end portion. Each of the main body 101 and the sub-body 102, the adjacent sub-bodies 102, and the sub-body 102 and the cap 103 is set to have diameters to ensure fitting. Note that, for miniaturizing the fluid resistor 100, the sub-body 102 and the cap 103 on the leading end side are fixed to hold the orifice films 110. However, the plurality of orifice films 110 may be held by the main body 101 and the plurality of sub-bodies 102 without the cap 103.

The orifice films 110 are held between the main body 101, the plurality of sub-bodies 102, and the cap 103. Additionally, the orifice film 110 has the orifice hole 110a in a region where the orifice hole 110a faces the hollow portions of the main body 101 and the sub-body 102. Each of the orifice holes 110a forms the flow rate resistor.

The fluid resistor 100 configured in this manner can be formed by disposing the plurality of orifice films 110 with the orifice holes 110a, which are the plurality of flow rate resistors (orifices) disposed in series, in series. Thus, the fluid resistor 100 can be miniaturized. The use of the fluid resistor 100 allows miniaturizing the blood pressure measurement device.

Note that the fluid resistor 100 is not limited to these configurations, and, for example, as disclosed in JP 2019-173796 A, each of the orifice holes adjacent along an axial direction may be formed to be shifted to one another in a radial direction. Additionally, as illustrated in FIG. 27 and FIG. 28, the orifice sheet 110 may be adhered to the distal end of the main body 101 formed in a hollow shape so as to ensure forming a flow path. The orifice sheet 110 having the orifice hole 110a of the fluid resistor 100 is, for example, integrally formed to a cylindrical base portion 104, or is adhered to the base portion 104. The base portion 104 is adhered to the main body 101. For example, the orifice sheet 110 and a base portion 104 are formed using various fine processing techniques, such as a laser processing technique. Note that as the fine processing technique, a technique similar to a processing technique used for Micro Electro Mechanical Systems (MEMS) can be used.

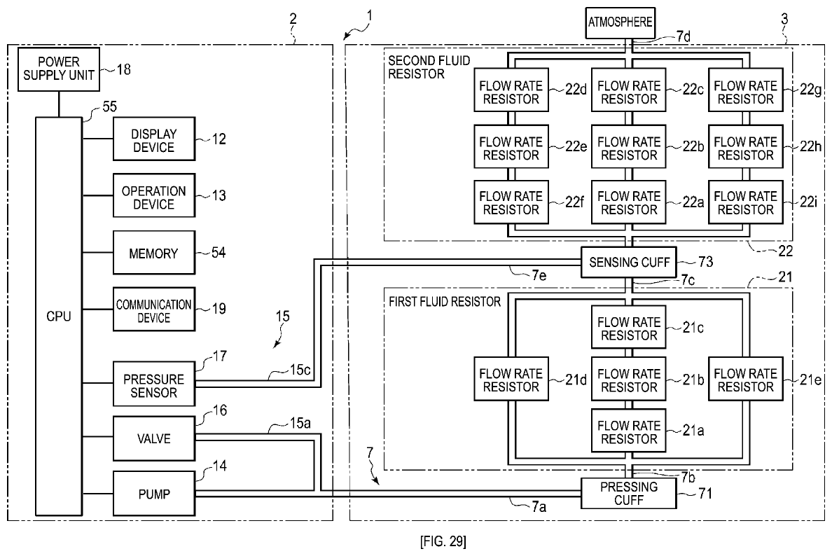

Also, FIG. 29 illustrates another modified example of the blood pressure measurement device 1 according to the twelfth embodiment in which a plurality of flow rate resistors are disposed in series and in parallel as an example of the blood pressure measurement device 1 including the first fluid resistor 21 and the second fluid resistor 22 using a plurality of orifices.

As illustrated in FIG. 29, the first fluid resistor 21 and the second fluid resistor 22 are configured by connecting a plurality of flow rate resistors in series and in parallel. The first fluid resistor 21 is an example in which, for example, the three flow rate resistors 21a, 21b, and 21c are connected in series and the respective flow rate resistors 21a, 21b, and 21c, single flow rate resistor 21d, and single flow rate resistor 21e are connected in parallel. The second fluid resistor 22 has a configuration in which, for example, the three flow rate resistors 22a, 22b, and 22c connected in series, the three flow rate resistors 22d, 22e, and 22f connected in series, and the three flow rate resistors 22g, 22h, and 22i connected in series are connected in parallel.

In this manner, in the case where the plurality of fluid resistors are connected in series and parallel, the plurality of orifice holes 110a may be provided in any or all of the plurality of orifice films 110 disposed between the main body 101, the plurality of sub-bodies 102, and the cap 103 as illustrated in FIG. 25, or the plurality of fluid resistors 100 may be connected in parallel as illustrated in FIG. 23.

As described in the twelfth embodiment and the modified example thereof, in at least one of the two or more fluid resistors used in the blood pressure measurement device, the plurality of orifices (flow rate resistors) are used in series, in parallel, or in series and parallel, thus ensuring performing differential pressure control while managing pressure dependence.

Note that the present invention is not limited to the respective embodiments described above. For example, in the cuff structure 6, the plurality of cuffs can be appropriately set, and the cuffs may be other than the pressing cuff, the sensing cuff, the tensile cuff, the winding cuff, or the measurement cuff described above.

Additionally, in the examples described above, the respective components of the fluid circuit 3 are not electrically controlled, and are controlled by the components provided outside the device body 2. However, the configuration is not limited thereto. That is, the fluid circuit 3 may be configured to further include the pump 14, the on-off valve 16, and the pressure sensor 17, in addition to the cuff structure 6, the tube group 7, and the fluid control units 9, 9A, and 9B as the configuration.

Furthermore, in view of miniaturizing the device body 2, the components of the fluid circuit 3 are preferably provided outside the device body 2, but obviously may be housed in the device body 2.

Note that the present invention is not limited to the above-described embodiments, and various modifications can be made in an implementation stage without departing from the gist. Further, embodiments may be carried out as appropriate in a combination, and combined effects can be obtained in such case. Further, the various inventions are included in the embodiment, and the various inventions may be extracted in accordance with combinations selected from the plurality of disclosed constituent elements. For example, in a case where the problem can be solved and the effects can be obtained even when some constituent elements are removed from the entire constituent elements given in the embodiment, the configuration obtained by removing the constituent elements may be extracted as an invention.

### REFERENCE NUMERALS LIST

1 Blood pressure measurement device
1A Blood pressure measurement device
1B Blood pressure measurement device
1C Blood pressure measurement device
1D Blood pressure measurement device
1E Blood pressure measurement device
1F Blood pressure measurement device
1G Blood pressure measurement device
1H Blood pressure measurement device
1I Blood pressure measurement device
2 Device body
2A Device body
3 Fluid circuit
3A Fluid circuit
3B Fluid circuit
3F Fluid circuit
3H Fluid circuit
3I Fluid circuit
4 Fixture (belt)
5 Curler
6 Cuff structure
7 Tube group
7a First tube
7b Second tube
7b1 Branch portion
7b2 Tube portion
7b3 Tube portion
7b4 Tube portion
7c Third tube
7c1 Branch portion
7c2 Tube portion
7c3 Tube portion
7c4 Tube portion
7c5 Tube portion
7d Fourth tube
7e Fifth tube
7f Sixth tube
9 Fluid control unit
9A Fluid control unit
9B Fluid control unit
11 Case
12 Display device
13 Operation device
14 Pump
15 Flow path unit
15a Flow path
15b Flow path
15c Flow path
16 On-off valve
17 Pressure sensor
18 Power supply unit
19 Communication device
20 Control substrate
21 First fluid resistor
21a Flow rate resistor (orifice)
21b Flow rate resistor (orifice)
21c Flow rate resistor (orifice)
21d Flow rate resistor (orifice)
21e Flow rate resistor (orifice)
22 Second fluid resistor
22a Flow rate resistor (orifice)
22b Flow rate resistor (orifice)
22c Flow rate resistor (orifice)
22d Flow rate resistor (orifice)
22e Flow rate resistor (orifice)
22f Flow rate resistor (orifice)
22g Flow rate resistor (orifice)
22h Flow rate resistor (orifice)
22i Flow rate resistor (orifice)
23 Valve (first valve)
24 Second valve
25 Third fluid resistor
25 Fourth fluid resistor
26 Third fluid resistor
27 Third valve
28 Fourth fluid resistor
29 Third valve
29I Third valve
31 Outer case
31a Lug
31b Spring rod
32 Windshield
41 Button
43 Touch panel
54 Storage unit
55 Control unit
55a Measurement processing unit
61 First belt
61a Belt portion
61b Buckle
62 Second belt
71 First cuff
72 Back plate
73 Second cuff
74 Third cuff
81 Air bag
84 Connection portion
91 Air bag
92 Flow path body
93 Connection portion
100 Fluid resistor
101 Main body
102 Sub-body
110 Orifice film
110 Orifice sheet
110a Orifice hole
200 Living body (wrist)
210 Artery

What is claimed is:

1. A fluid circuit, comprising:
a first cuff connected to a secondary side of a pump that supplies a fluid to a secondary side;
a first fluid resistor connected to a secondary side of the first cuff;
a second fluid resistor provided on a secondary side of the first fluid resistor and connected to an atmosphere; and
a second cuff provided between the first fluid resistor and the second fluid resistor.

2. The fluid circuit according to claim 1, comprising
a first valve provided in parallel with the first fluid resistor, the first valve opening when a pressure of the second cuff is higher than a pressure of the first cuff by a predetermined value or more.

3. The fluid circuit according to claim 1, comprising:
a second valve provided in parallel with the first fluid resistor, the second valve opening when a pressure of the first cuff is higher than a pressure of the second cuff by a predetermined value or more; and
a third fluid resistor connected to a secondary side of the second valve, the third fluid resistor being provided in parallel with the first fluid resistor.

4. The fluid circuit according to claim 1, wherein
at least one of the first fluid resistor and the second fluid resistor is formed by connecting a plurality of orifices in parallel, in series, or in series and parallel.

5. A blood pressure measurement device, comprising:
a pump that supplies a fluid to a secondary side;
the fluid circuit according to claim 1;
an on-off valve provided between the pump and the first cuff, the on-off valve opening and closing a flow path to the atmosphere;
a pressure sensor connected to the second cuff; and
a control unit that controls the pump and the on-off valve based on a pressure detected by the pressure sensor.

6. The blood pressure measurement device according to claim 5, comprising
a device body that houses the pump, the on-off valve, the pressure sensor, and the control unit, wherein
the first fluid resistor and the second fluid resistor are integrally provided with the first cuff.

7. The fluid circuit according to claim 2, comprising:
a second valve provided in parallel with the first fluid resistor, the second valve opening when a pressure of the first cuff is higher than a pressure of the second cuff by a predetermined value or more; and
a third fluid resistor connected to a secondary side of the second valve, the third fluid resistor being provided in parallel with the first fluid resistor.

8. The fluid circuit according to claim 2, wherein
at least one of the first fluid resistor and the second fluid resistor is formed by connecting a plurality of orifices in parallel, in series, or in series and parallel.

9. The fluid circuit according to claim 3, wherein
at least one of the first fluid resistor and the second fluid resistor is formed by connecting a plurality of orifices in parallel, in series, or in series and parallel.

10. The fluid circuit according to claim 7, wherein
at least one of the first fluid resistor and the second fluid resistor is formed by connecting a plurality of orifices in parallel, in series, or in series and parallel.

11. A blood pressure measurement device, comprising:
a pump that supplies a fluid to a secondary side;
the fluid circuit according to claim 2;
an on-off valve provided between the pump and the first cuff, the on-off valve opening and closing a flow path to the atmosphere;
a pressure sensor connected to the second cuff; and
a control unit that controls the pump and the on-off valve based on a pressure detected by the pressure sensor.

12. A blood pressure measurement device, comprising:
a pump that supplies a fluid to a secondary side;
the fluid circuit according to claim 3;
an on-off valve provided between the pump and the first cuff, the on-off valve opening and closing a flow path to the atmosphere;
a pressure sensor connected to the second cuff; and
a control unit that controls the pump and the on-off valve based on a pressure detected by the pressure sensor.

13. A blood pressure measurement device, comprising:
a pump that supplies a fluid to a secondary side;
the fluid circuit according to claim 4;
an on-off valve provided between the pump and the first cuff, the on-off valve opening and closing a flow path to the atmosphere;
a pressure sensor connected to the second cuff; and
a control unit that controls the pump and the on-off valve based on a pressure detected by the pressure sensor.

14. A blood pressure measurement device, comprising:
a pump that supplies a fluid to a secondary side;
the fluid circuit according to claim 7;
an on-off valve provided between the pump and the first cuff, the on-off valve opening and closing a flow path to the atmosphere;
a pressure sensor connected to the second cuff; and
a control unit that controls the pump and the on-off valve based on a pressure detected by the pressure sensor.

15. A blood pressure measurement device, comprising:
a pump that supplies a fluid to a secondary side;
the fluid circuit according to claim 8;
an on-off valve provided between the pump and the first cuff, the on-off valve opening and closing a flow path to the atmosphere;
a pressure sensor connected to the second cuff; and
a control unit that controls the pump and the on-off valve based on a pressure detected by the pressure sensor.

16. A blood pressure measurement device, comprising:
a pump that supplies a fluid to a secondary side;
the fluid circuit according to claim 9;
an on-off valve provided between the pump and the first cuff, the on-off valve opening and closing a flow path to the atmosphere;
a pressure sensor connected to the second cuff; and
a control unit that controls the pump and the on-off valve based on a pressure detected by the pressure sensor.

17. A blood pressure measurement device, comprising:
a pump that supplies a fluid to a secondary side;
the fluid circuit according to claim 10;
an on-off valve provided between the pump and the first cuff, the on-off valve opening and closing a flow path to the atmosphere;
a pressure sensor connected to the second cuff; and
a control unit that controls the pump and the on-off valve based on a pressure detected by the pressure sensor.
